(19) United States
(12) Patent Application Publication
(10) Pub. No.: US 2024/0263132 A1
(43) Pub. Date: Aug. 8, 2024

(54) RECOMBINANT EXPRESSION OF KLEBSIELLA PNEUMONIAE O-ANTIGENS IN ESCHERICHIA COLI

(71) Applicant: Pfizer Inc., New York, NY (US)

(72) Inventors: Robert George Konrad Donald, South Orange, NJ (US); Aniruddha Sasmal, Westwood, NJ (US)

(21) Appl. No.: 18/562,387

(22) PCT Filed: May 23, 2022
(86) PCT No.: PCT/IB2022/054808

§ 371 Date: Nov. 20, 2023

Related U.S. Application Data
(60) Provisional application No. 63/193,124, filed on May 26, 2021.

(51) Int. Cl. C12N 1/20; C07K 14/26; C12N 9/10; C12N 9/90; C12N 15/52; C12N 15/70; C12R 1/19

(52) U.S. Cl. CPC C12N 1/205; C07K 14/26; C12N 9/1051; C12N 9/90; C12N 15/52; C12N 15/70; C12R 2001/19; C12Y 504/99009

(57) ABSTRACT

This invention provides a recombinant Escherichia coli (E. coli) host cell for producing a Klebsiella pneumoniae (K. pneumoniae) O-antigen, wherein the E. coli host cell comprises a polynucleotide encoding the K. pneumoniae O-antigen, including methods of producing and purifying the K. pneumoniae O-antigen.

### CROSS REFERENCE TO RELATED APPLICATIONS

This application claims the benefits of U.S. Provisional Application No. 63/193,124, filed May 26, 2021, the entire content of which is incorporated herein by reference in its entirety.

### REFERENCE TO SEQUENCE LISTING

This application is being filed electronically via EFS-Web and includes an electronically submitted sequence listing in .txt format. The .txt file contains a sequence listing entitled “PC072734_SequenceListing_26April2022_ST25.txt” created on Apr. 26, 2022 and having a size of 71 KB. The sequence listing contained in this .txt file is part of the specification and is incorporated herein by reference in its entirety.

### FIELD OF THE INVENTION

The present invention relates to an E. coli platform for the expression of Klebsiella pneumoniae O-antigens.

### BACKGROUND OF THE INVENTION

Multidrug-resistant Klebsiella pneumoniae infections are an increasing cause of mortality in vulnerable populations at risk. The O1 and O2 O-antigen serotypes are highly prevalent among strains causing invasive disease globally and derived O-antigen glycoconjugates are attractive as vaccine antigens. The O1 and O2 O-antigens and their corresponding v1 and v2 subtypes are polymeric galactans that differ in the structures of their repeat units. Purification of native O-antigens from Klebsiella clinical strains is complicated by the co-expression of high levels of other surface polysaccharides which contributes to a high degree of viscosity during fermentation and consequently reduces the efficiency of downstream bioprocesses.

Accordingly, there exists a need for improved methods of producing O-antigen serotypes of Klebsiella pneumoniae, especially the O1 and O2 serotypes.

### SUMMARY OF THE INVENTION

This invention provides a recombinant Escherichia coli (E. coli) host cell for producing a Klebsiella pneumoniae (K. pneumoniae) O-antigen, wherein the E. coli host cell comprises a polynucleotide encoding the K. pneumoniae O-antigen.

In a first embodiment, the K. pneumoniae O-antigen is selected from serotype O1 or serotype O2. In one aspect of this embodiment, the K. pneumoniae O-antigen is selected from subtype v1 or subtype v2. In another aspect of this embodiment, the K. pneumoniae O-antigen is selected from the group consisting of:
a) serotype O1 subtype v1 (O1v1),
b) serotype O1 subtype v2 (O1v2),
c) serotype O2 subtype v1 (O2v1), and
d) serotype O2 subtype v2 (O2v2).

In a second embodiment, the recombinant E. coli host cell is an E. coli O-antigen mutant strain. In one aspect of this embodiment, the E. coli host cell is an E. coli K12 strain.

In a third embodiment, the polynucleotide sequence further encodes one or more primers.

In a fourth embodiment, the polynucleotide is integrated into a vector.

In a fifth embodiment, the polynucleotide is integrated into the genomic DNA of the E. coli cell.

In a sixth embodiment, the polynucleotide comprises nucleotides encoding a gene cluster that is at least 75%, 80%, 85%, 90%, 95%, 96%, 97%, 98%, or 99% identical to SEQ ID NOs: 13-15 and 16-25 or a combination thereof.

This invention also provides a vector comprising a polynucleotide encoding a K. pneumoniae O-antigen. In one aspect, the K. pneumoniae O-antigen is selected from serotype O1 or serotype O2. In another aspect, the K. pneumoniae O-antigen is selected from subtype v1 or subtype v2. In another aspect, the K. pneumoniae O-antigen is selected from the group consisting of: a) serotype O1 subtype v1 (O1v1), b) serotype O1 subtype v2 (O1v2), c) serotype O2 subtype v1 (O2v1), and d) serotype O2 subtype v2 (O2v2).

This invention also provides a culture comprising the recombinant E. coli host cell described hereinabove, wherein said culture is at least 5 liters in size.

This invention further provides a method for producing a K. pneumoniae O-antigen, comprising
a. culturing a recombinant E. coli host cell according to claim 1 under a suitable condition, thereby expressing the K. pneumoniae O-antigen; and
b. harvesting the K. pneumoniae O-antigen produced by step (a).

In one aspect, the method further comprises a step for purifying the K. pneumoniae O-antigen.

### BRIEF DESCRIPTION OF THE DRAWINGS

FIG. 1 depicts the carbohydrate repeat unit structures of the predominant Klebsiella serotype O1 and O2 O-antigen subtypes. Structures of the base galactans I and III that define the two distinct serotype O2 subtypes O2v1 and O2v2 are shown in the left panels. Derived chimeras resulting from capping by galactan II, which is the immunodominant determinant for serotype O1, yields subtypes O1v1 and O1v2 that are shown in the right panels (see Kelly S D, et al. J Biol Chem 2019; 294:10863-76; Clarke B R, et al. J Biol Chem 2018; 293:4666-79).

FIG. 2A-2B depict the Klebsiella pneumoniae O2 O-antigen galactan I and galactan III biosynthetic gene clusters. FIG. 2A shows the structure of the v1 gene cluster responsible for galactan I biosynthesis from strain PFEKP0011. FIG. 2B shows the structure of the v2 gene cluster responsible for galactan III biosynthesis from strain PFEKP0049. Primers S2 and AS2 were used to amplify the respective 8.2 kb and 11.1 kb fragments from different Klebsiella strains for cloning into pBAD vectors. Genes gmIABC present at the 3′ end of the v2 gene cluster encode enzymes that transfer a galactose side chain to the galactose disaccharide repeat unit converting galactan I (O2v1) to galactan III (O2v2) (see FIG. 1).

FIG. 3 depicts the expression of galactan I and III LPS in E. coli v1 or v2 plasmid transformants. Experimental details: LPS was extracted from plasmid transformants of E. coli K12 strain BD643 ΔwzzB grown in 3 mL LB cultures in the presence or absence of 0.2% arabinose. Samples were resolved on a Criterion 4-12% SDS-PAGE gel (Biorad) and carbohydrate detected with Emerald 300 stain (Thermo). E. coli O55 LPS was run as a control. Empty vector (EV) is the pBAD33 plasmid with no insert. M is a protein molecular mass Kaleidoscope™ standard. Plasmid clone numbers, gene cluster type (v1 or v2) and inferred galactans are indicated (see Table 4).

FIG. 4 depicts Klebsiella pneumoniae O1 O-antigen galactan II gene cluster. The structure of the wbby-wbbyz locus responsible for galactan II biosynthesis cloned from strain PFEKP0011 is shown. Primers PCRS1 and PCRAS1 were used to PCR amplify the 3.4 kb fragment from representative Klebsiella strains for cloning into the pTopo vector. Flanking genes are putative transposase-encoding genes that are likely not associated with the biosynthesis of LPS (Hsieh P-F, et al. Frontiers in Microbiology 2014; 5: 608).

FIG. 5 depicts the expression of chimeric Klebsiella II-I and II-III galactans by combining v1 or v2 operon plasmids with compatible wbbzy plasmids in E. coli. Experimental details are common to FIG. 3. In this case plasmid transformants were grown in the absence of arabinose inducer. P—parental clones 1-2 and 8-2 harboring respective v1 and v2 operons cloned from O1v1 and O1v2 Klebsiella strains PFEKP0011 and PFEKP0049 (see also Table 4). Clones 211-214 and clones 821-824 are four independent double transformants of these parents harboring an additional Topo plasmid containing wbbzy genes cloned from the homologous Klebsiella strain.

FIG. 6 depicts small scale purification of recombinant Klebsiella O1 and O2 O-antigens. A primary workflow of small scale culture, purification, and characterization of recombinant Klebsiella O-antigen is described in this figure. The growth conditions are described in Table 5. After harvesting the bacteria, O-antigen was extracted by acid hydrolysis and purified by ultra filtration and membrane chromatography. Characterization was done by NMR, HPAEC-PAD, and SEC-MALLS analysis.

FIGS. 7A and 7B depict HPLC (Refractive Index Detection) profiles of purified recombinant Klebsiella O-antigens. These figures depict representative HPLC chromatograms of purified recombinant Klebsiella O-antigens: O1V1 and O1V2 (FIG. 7A), and O2V1 AND 02V2 (FIG. 7B). HPLC conditions include isocratic PBS gradient, size-exclusion column, and refractive index detector to monitor the sample purity. O-antigen profiles showed significantly pure sample was obtained.

FIG. 8 depicts ¹H-NMR profiles which confirm distinct chemical shifts of anomeric protons. ¹H-NMR of purified O-antigen was recorded and the anomeric region displayed distinct chemical shifts of the corresponding galactose unit present in the repeating unit of the polysaccharide. The peak annotations were based on the 1D and 2D NMR, and also comparing to the reported literature values (Vinogradov J. Biol. Chem. 2002, 277, 25070-25081). The normalized peak integration values confirmed ˜2:1 ratio between the chain length of Galactan II vs. Galactan I/III in O1 subtype antigens.

FIG. 9A-9C depict coupled HSQC which confirm linkage stereochemistry. Proton-coupled HSQC spectra was recorded for O1v1 (FIG. 9C), O2v1 (FIG. 9A), and O2v2 (FIG. 9B) to identify the anomeric stereochemistry. For the galactopyranose structures, coupling constant greater than 169 Hz generally indicates an alpha connection whereas the value smaller than 169 Hz indicates a beta linkage. Due to the puckered five-membered ring structure the furanose anomeric proton-carbon coupling values differ significantly. Here the beta-linked galactofuranose anomeric center showed a coupling constant of ˜173 Hz.

FIG. 10 shows that NMR chemical shifts agree with values reported for native Klebsiella O-antigens. The chemical shift difference (CSD) was calculated using the formula CSD=√(δH2+0.3*δC2), where δH and δC are the differences between the reported ppm and the experimental ppm values in proton and carbon NMR respectively. CSD value below 0.2 indicates a good match with the reported structure.

### SEQUENCE IDENTIFIERS

SEQ ID NO: 1 sets forth the amino acid sequence of Transport permease protein (wzm);
SEQ ID NO: 2 sets forth the amino acid sequence of ABC transporter, ATP-binding component (wzt);
SEQ ID NO: 3 sets forth the amino acid sequence of Glycosyltransferase (wbbM);
SEQ ID NO: 4 sets forth the amino acid sequence of UDP-galactopyranose mutase (glf);
SEQ ID NO: 5 sets forth the amino acid sequence of Galactosyltransferase (wbbN);
SEQ ID NO: 6 sets forth the amino acid sequence of Galactosyltransferase (wbbO);
SEQ ID NO: 7 sets forth the amino acid sequence of FGlycosyltransferase family 2 (kfoC);
SEQ ID NO: 8 sets forth the amino acid sequence of GmIC protein;
SEQ ID NO: 9 sets forth the amino acid sequence of GmIB protein;
SEQ ID NO: 10 sets forth the amino acid sequence of GmIA protein;
SEQ ID NO: 11 sets forth the amino acid sequence of Glycosyltransferase (wbbY);
SEQ ID NO: 12 sets forth the amino acid sequence for Exopolysaccharide biosynthesis protein (wbbZ);
SEQ ID NO: 13 sets forth the nucleic acid sequence for the 8.2 kb v1 operon fragment (Gal I biosynthetic gene cluster);
SEQ ID NO: 14 sets forth the nucleic acid sequence for the 11.1 kb v2 operon (Gal III biosynthetic gene cluster);
SEQ ID NO: 15 sets forth the nucleic acid sequence for the 3.4 kb wbbZY fragment (Gal II biosynthetic gene cluster); 30
SEQ ID NO: 16 sets forth the nucleic acid sequence of the oligonucleotide primer wzm5′S2; SEQ ID NO: 17 sets forth the nucleic acid sequence of the oligonucleotide primer his13′AS2;
SEQ ID NO: 18 sets forth the nucleic acid sequence of the oligonucleotide primer wzm5′S3; SEQ ID NO: 19 sets forth the nucleic acid sequence of the oligonucleotide primer his13′AS3;
SEQ ID NO: 20 sets forth the nucleic acid sequence of the oligonucleotide primer pBAD33_O1O2S;
SEQ ID NO: 21 sets forth the nucleic acid sequence of the oligonucleotide primer pBAD33_O1O2AS;
SEQ ID NO: 22 sets forth the nucleic acid sequence of the oligonucleotide primer pBAD18_O1O2S;
SEQ ID NO: 23 sets forth the nucleic acid sequence of the oligonucleotide primer pBAD18 O102AS;
SEQ ID NO: 24 sets forth the nucleic acid sequence of the oligonucleotide primer wbbZY PCR S1; and
SEQ ID NO: 25 sets forth the nucleic acid sequence of the oligonucleotide primer wbbZY PCR AS1.

### DETAILED DESCRIPTION OF THE INVENTION

This invention overcomes the challenges encountered with production of Klebsiella pneumoniae O1 and O2 O-antigens in Klebsiella clinical strains by expressing these antigens in E. coli for the first time.

This invention provides a recombinant Escherichia coli (E. coli) host cell for producing a Klebsiella pneumoniae (K. pneumoniae) O-antigen, wherein the E. coli host cell comprises a polynucleotide encoding the K. pneumoniae O-antigen.

In a first embodiment, the K. pneumoniae O-antigen is selected from serotype O1 or serotype O2. In one aspect of this embodiment, the K. pneumoniae O-antigen is selected from subtype v1 or subtype v2. In another aspect of this embodiment, the K. pneumoniae O-antigen is selected from the group consisting of:
a) serotype O1 subtype v1 (O1v1),
b) serotype O1 subtype v2 (O1v2),
c) serotype O2 subtype v1 (O2v1), and
d) serotype O2 subtype v2 (O2v2).

In another aspect, the polynucleotide encoding the K. pneumoniae O2v1 O-antigen comprises a gene cluster, wherein the gene cluster encodes:
a. Transport permease protein,
b. ABC transporter, ATP-binding component,
c. Glycosyltransferase,
d. UDP-galactopyranose mutase,
e. Galactosyltransferase (encoded by both wbbN and wbbO), and
f. FGlycosyltransferase family 2.

In another aspect, the polynucleotide encoding the K. pneumoniae O2v2 O-antigen comprises a gene cluster, wherein the gene cluster encodes:
a. Transport permease protein,
b. ABC transporter, ATP-binding component,
c. Glycosyltransferase,
d. UDP-galactopyranose mutase,
e. Galactosyltransferase (encoded by both wbbN and wbbO),
f. FGlycosyltransferase family 2,
g. protein encoded by gmIC (galactosyltransferase),
h. GmIB protein, and
i. GmIA protein.

In another aspect, the polynucleotide encoding the K. pneumoniae O1v1 O-antigen comprises:
a. a first gene cluster, wherein the first gene cluster encodes
   i. Transport permease protein,
   ii. ABC transporter, ATP-binding component,
   iii. Glycosyltransferase,
   iv. UDP-galactopyranose mutase,
   v. Galactosyltransferase (encoded by both wbbN and wbbO), and
   vi. FGlycosyltransferase family 2;
and
b. a second gene cluster, wherein the second gene cluster encodes
   i. glycosyltransferase, and
   ii. exopolysaccharide biosynthesis protein.

In another aspect, the polynucleotide encoding the K. pneumoniae O1v2 O-antigen comprises:
a. a first gene cluster, wherein the first gene cluster encodes
   i. a. Transport permease protein,
   ii. ABC transporter, ATP-binding component,
   iii. Glycosyltransferase,
   iv. UDP-galactopyranose mutase,
   v. Galactosyltransferase (encoded by both wbbN and wbbO?),
   vi. FGlycosyltransferase family 2,
   vii. protein encoded by gmIC (please provide name),
   viii. GmIB protein, and
   ix. GmIA protein;
and
b. a second gene cluster, wherein the second gene cluster encodes
   i. glycosyltransferase, and
   ii. exopolysaccharide biosynthesis protein.

In another aspect, the polynucleotide encoding the K. pneumoniae O2v1 O-antigen comprises a gene cluster, wherein the gene cluster comprises the K. pneumoniae genes:
a. wzm,
b. wzt,
c. wbbM,
d. gif,
e. wbbN,
f. wbbO, and
g. kfoC.

In another aspect, the polynucleotide encoding the K. pneumoniae O2v2 O-antigen comprises a gene cluster, wherein the gene cluster comprises the K. pneumoniae genes:
a. wzm,
b. wzt,
c. wbbM,
d. glf,
e. wbbN,
f. wbbO,
g. kfoC,
h. gmIC,
i. gmIB, and
j. gmIA.

In another aspect, the polynucleotide encoding the K. pneumoniae O1v1 O-antigen comprises:
a. a first gene cluster, wherein the first gene cluster comprises the K. pneumoniae genes:
   i. wzm,
   ii. wzt,
   iii. wbbM,
   iv. gif,
   v. wbbN,
   vi. wbbO,
   vii. kfoC;
and
b. a second gene cluster, wherein the second gene cluster comprises the K. pneumoniae genes:
   i. wbbY, and
   ii. wbbZ.

In another aspect, the polynucleotide encoding the K. pneumoniae O1v2 O-antigen comprises:
a. a first gene cluster, wherein the first gene cluster comprises the K. pneumoniae genes:
   i. wzm,
   ii. wzt,
   iii. wbbM,
   iv. gif,
   v. wbbN,
   vi. wbbO,
   vii. kfoC,
   viii. gmIC,
   ix. gmIB, and
   x. gmIA;
and
b. a second gene cluster, wherein the second gene cluster comprises the K. pneumoniae genes:
   i. wbbY, and
   ii. wbbZ.

In another aspect, the polynucleotide encoding the K. pneumoniae O2v1 O-antigen comprises a gene cluster, wherein the gene cluster comprises nucleotides having the nucleotide sequence set forth in SEQ ID NO: 13.

In another aspect, the polynucleotide encoding the K. pneumoniae O2v2 O-antigen comprises a gene cluster, wherein the gene cluster comprises nucleotides having the nucleotide sequence set forth in SEQ ID NO: 14.

In another aspect, the polynucleotide encoding the K. pneumoniae O1v1 O-antigen comprises:
a. a first gene cluster, wherein the first gene cluster comprises nucleotides having the nucleotide sequence set forth in SEQ ID NO: 13; and
b. a second gene cluster, wherein the second gene cluster comprises nucleotides having the nucleotide sequence set forth in SEQ ID NO: 15.

In another aspect, the nucleotide encoding the K. pneumoniae O1v2 O-antigen comprises:
a. a first gene cluster, wherein the first gene cluster comprises nucleotides having the nucleotide sequence set forth in SEQ ID NO: 14; and
b. a second gene cluster, wherein the second gene cluster comprises nucleotides having the nucleotide sequence set forth in SEQ ID NO: 15.

In another aspect, the polynucleotide encoding the K. pneumoniae O2v1 O-antigen comprises a gene cluster, wherein the gene cluster comprises nucleotides encoding the polypeptides having the amino acid sequences set forth in SEQ ID NOS: 1-7 or a fragment thereof.

In another aspect, the polynucleotide encoding the K. pneumoniae O2v2 O-antigen comprises a gene cluster, wherein the gene cluster comprises nucleotides encoding the polypeptides having the amino acid sequences set forth in SEQ ID NOs: 1-10 or a fragment thereof.

In another aspect, the polynucleotide encoding the K. pneumoniae O1v1 O-antigen comprises:
a. a first gene cluster, wherein the first gene cluster comprises nucleotides encoding the polypeptides having the amino acid sequences set forth in SEQ ID NOs: 1-7 or a fragment thereof; and
b. a second gene cluster, wherein the second gene cluster comprises nucleotides encoding the polypeptides having the amino acid sequences set forth in SEQ ID NOs: 11-12 or a fragment thereof.

In another aspect, the polynucleotide encoding the K. pneumoniae O1v2 O-antigen comprises:
a. a first gene cluster, wherein the first gene cluster comprises nucleotides encoding the polypeptides having the amino acid sequences set forth in SEQ ID NOs: 1-10; and
b. a second gene cluster, wherein the second gene cluster comprises nucleotides encoding the polypeptides having the amino acid sequences set forth in SEQ ID NOs: 11-12.

In a second embodiment, the recombinant E. coli host cell is an E. coli O-antigen mutant strain. In one aspect of this embodiment, the E. coli host cell is an E. coli K12 strain.

In a third embodiment, the polynucleotide sequence further encodes one or more primers. In one aspect, the primer comprises at least 25 nucleic acid residues and at most 100 nucleic acid residues. In another aspect, the primer comprises nucleic acids having the sequence selected from the group consisting of:
a. SEQ ID NO: 16 (wzm5′S2);
b. SEQ ID NO: 17 (hisl3′AS2);
c. SEQ ID NO: 18 (wzm5′S3);
d. SEQ ID NO: 19 (hisl3′AS3);
e. SEQ ID NO: 20 (pBAD33_O1O2S);
f. SEQ ID NO: 21 (pBAD33_O1O2AS);
g. SEQ ID NO: 22 (BAD18_O1O2S);
h. SEQ ID NO: 23 (pBAD18_O1O2AS);
i. SEQ ID NO: 24 (wbbZY PCR S1); and
j. SEQ ID NO: 25 (wbbZY PCR AS1).

In a fourth embodiment, the polynucleotide is integrated into a vector. In one aspect, the vector is a plasmid. In another aspect, the plasmid is selected from the group consisting of:
a. pBAD33;
b. pBAD18; and
c. Topo-blunt II.

In a fifth embodiment, the polynucleotide is integrated into the genomic DNA of the E. coli cell. In one aspect, the polynucleotide is codon optimized for expression in the E. coli cell.

In a sixth embodiment, the polynucleotide comprises nucleotides encoding a gene cluster that is at least 75%, 80%, 85%, 90%, 95%, 96%, 97%, 98%, or 99% identical to SEQ ID NOs: 13-15 and 16-25 or a combination thereof.

This invention also provides a vector comprising a polynucleotide encoding a K. pneumoniae O-antigen. In one aspect, the K. pneumoniae O-antigen is selected from serotype O1 or serotype O2. In another aspect, the K. pneumoniae O-antigen is selected from subtype v1 or subtype v2. In another aspect, the K. pneumoniae O-antigen is selected from the group consisting of: a) serotype O1 subtype v1 (O1v1), b) serotype O1 subtype v2 (O1v2), c) serotype O2 subtype v1 (O2v1), and d) serotype O2 subtype v2 (O2v2).

In a further aspect, the vector is a plasmid. In another aspect, the plasmid is selected from the group consisting of:
a. pBAD33;
b. pBAD18; and
c. Topo-blunt II.

This invention also provides a culture comprising the recombinant E. coli host cell described in the embodiments hereinabove, wherein said culture is at least 5 liters in size.

This invention further provides a method for producing a K. pneumoniae O-antigen, comprising
a. culturing a recombinant E. coli host cell according to the embodiments described hereinabove under a suitable condition, thereby expressing the K. pneumoniae O-antigen; and
b. harvesting the K. pneumoniae O-antigen produced by step (a).

In one aspect, the method further comprises a step for purifying the K. pneumoniae O-antigen.

Those skilled in the art will appreciate that due to the degeneracy of the genetic code, a protein having a specific amino acid sequence can be encoded by multiple different nucleic acids. Thus, those skilled in the art will understand that a nucleic acid provided herein can be altered in such a way that its sequence differs from a sequence provided herein, without affecting the amino acid sequence of the protein encoded by the nucleic acid.

### EXAMPLES

In order that this invention may be better understood, the following examples are set forth. These examples are for purposes of illustration only and are not to be construed as limiting the scope of the invention in any manner. The following Examples illustrate some embodiments of the invention.

### Example 1

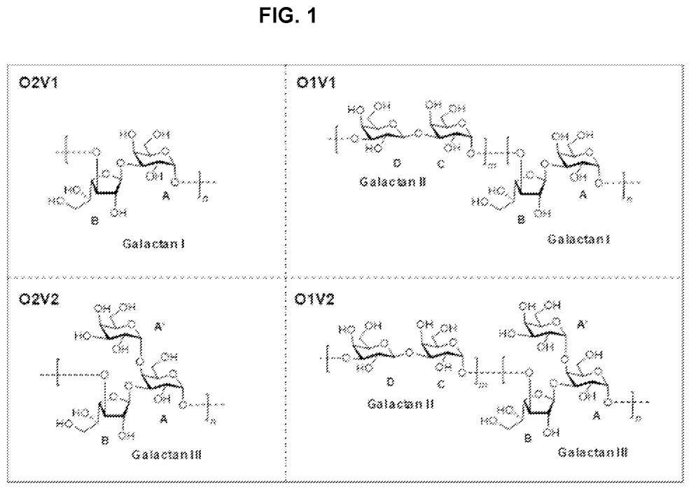

The genetic and structural basis for the expression of the major O-antigen subtypes of O1 and O2 (O1v1, O1v2, O2v1 and O2v2) was recently determined by Chris Whitfield's research group at U. Guelph, Canada (Kelly S D, et al. J Biol Chem 2019; 294:10863-76; Clarke B R, et al. J Biol Chem 2018; 293:4666-79). The structural relationships between the O-antigens which comprise these four subtypes are illustrated in FIG. 1. The four subtypes are all derived from the base galactan I polymer with its disaccharide repeat structure, the biosynthesis of which is controlled by the O2v1 gene cluster. The O2v2 gene cluster is the same as O2v1 except for the presence of three additional genes (gmlABC) at the 3′ end, whose encoded enzymes add a galactose side chain to each galactan I disaccharide repeat to generate the branched galactan III structure. Additional modifications to the O2v1 (galactan I) and O2v2 (galactan III) O-antigens involve addition of a second glycan repeat-unit structure, galactan II, to their nonreducing termini to produce the respective chimeric glycan II-I and glycan II-III O-antigens. Capping of the base O2v1 (galactan I) or O2v2 (galactan III) O-antigens by galactan II is mediated by enzymes encoded by the genes wbbY and wbbZ at an unlinked chromosomal locus (Kelly S D, et al. J Biol Chem 2019; 294:10863-76; Hsieh P-F, et al. Frontiers in microbiology 2014; 5:608).

The inventors used a modular approach. whereby expression of serotype O2 base galactans I and III was mediated by respective v1 or v2 gene clusters on p15a plasmids, with additional capping by galactan II to generate the corresponding serotype O1v1 and O1v2 chimeras conferred by coexpression of wbbzy genes from a second compatible CoIE1 plasmid.

First, serotype O2 subtypes comprised of homopolymeric and branched galactans were generated by cloning respective variant 1 and variant 2 gene clusters in a modified pBAD33 plasmid (p15a replicon) designed to accept long PCR fragments using the high fidelity Gibson reaction (NEB HiFi DNA assembly mix). Next, capping of these O-antigens with O1 specific galactan was achieved by co-expression of wbbzy genes cloned into the Topo-blunt II vector (high copy CoIE1 replicon), which is fully compatible with the recombinant pBAD33 plasmids.

Initial proof of concept for the heterologous expression of these O-antigens was successfully established at shake-flask scale. O-antigens were isolated by acid hydrolysis and purified by multiple purification steps (UFDF, Ion-exchange, hydrophobic interaction). Purified O1v1, O2v1 and O2v2 O-antigens thus obtained were characterized by analytical methods (NMR, HPAEC-PAD, SEC-MALS); 1-D and 2-D NMR showed proton and carbon peaks that matched published structures of the corresponding native Klebsiella galactans, confirming linkages and stereochemistry. Finally, the structure of the fourth O-antigen O1v2, obtained at lower yield than the others, was confirmed by ¹H-NMR.

The details of this work is set forth below:

### I. Materials and Methods

Nucleotide sequence information from Klebsiella O-antigen biosynthetic gene clusters was retrieved by BLAST searching whole genome sequence (WGS) assemblies. DNA fragment libraries were prepared from bacterial genomic DNA using a Nextera DNA Library kit and sequenced on a MiSeq instrument (Illumina). De novo assembly of short sequence reads was done with the CLC workbench software (Qiagen).

A. E. coli Host Strains

E. coli K12 lab strains are naturally deficient in O-antigen expression due to genetic insertion or deletion mutations in their O-antigen biosynthetic gene cluster (Liu D, Reeves P R. Microbiology (Reading) 1994; 140 (Pt 1):49-57). This feature makes the K12 strain or other E. coli O-antigen mutant strains useful for the expression of heterologous Klebsiella O-antigens (Izquierdo L, et al. Journal of bacteriology 2003; 185:1634-1641). For our exploratory work we initially used a commercial K12 host, and subsequently two E. coli strains generated in-house: a K12 host and an E. coli serotype O25b strain lacking its O-antigen biosynthetic gene cluster (Table 1). Both strains, BD643 DwzzB and PFEEC0100 OAg-, also harbor a deletion in the gene for the wzzB chain length regulator to prevent potential expression of endogenous O-antigens. All strains shown in Table 1 are O-antigen minus mutants (rough mutants) and do not express O-antigens or capsular antigens.

TABLE 1
E. coli Host Strains

| Strain ID | Genotype |
|---|---|
| NEB5α | fhuA2 Δ(argF-lacZ)U169 phoA glnV44 |
| | φ80Δ(lacZ)M15 gyrA96 |
| BD591 | F-, lambda-, IN(rrnD-rrnE)1, rph-1 |
| BD643 | BD591 DE3 ΔrecA ΔfhuA ΔaraA |
| BD643 ΔwzzB | BD591 DE3 ΔrecA ΔfhuA ΔaraA, ΔwzzB |
| PFEEC0100 OAg- | D(rflB-orf11)::tetRA ΔAraA ΔwzzB |

B. Klebsiella pneumoniae Clinical Strains

Urinary tract infection (UTI) isolates were obtained from the Pfizer-sponsored Antimicrobial Testing Leadership and Surveillance (ATLAS) collection, which is maintained by the International Health Management Associates (IHMA) clinical lab. In-silico serotyping of WGS data for the prediction of O-antigen and K-capsule types was done using the Kaptiveweb algorithm (Wick R R, et al. J Clin Microbiol 2018; 56), and multilocus sequence type (MLST-ST) determining according the Pasteur institute scheme (Diancourt L, et al. Journal of clinical microbiology 2005; 43:4178-82). Isolates from which O-antigen gene clusters were cloned are summarized in Table 2.

TABLE 2
Klebsiella pneumoniae Clinical Isolates used as the
Source of Galactan Biosynthetic Genes

| IHMA | Pfizer | MLST | Serotype | Galactan(s) | |
|---|---|---|---|---|---|
| Isolate | ID | ST | (subtype) | expressed | Source |
| 911202 | PFEKP0011 | 14 | O1(v1) | II-I | UTI, |
| | | | | | kidneys |
| 837643 | PFEKP0004 | 20 | O1(v2) | II-III | UTI, |
| | | | | | bladder |
| 837645 | PFEKP0005 | 337 | O2(v1) | I | UTI, |
| | | | | | bladder |
| 1508488 | PFEKP0049 | 416 | O1(v2) | II-III | UTI, |
| | | | | | bladder |
| 976438 | PFEKP0017 | 17 | O2(v2) | III | UTI, |
| | | | | | urethra |

### C. Molecular Cloning of O-Antigen Gene Clusters

Relevant O-antigen gene clusters were extracted based on homology with reference serotype O1 and O2 rfb operons, which are located at a chromosomal locus between gene clusters for K-capsule and histidine biosyntehsis (Follador R, et al. Microbial Genomics 2016; 2: e000073). Conserved PCR primers homologous to the first wzm (ABC permease) gene in rfb gene cluster and the 3′ flanking his/gene were designed to amplify v1 or v2 operon variants from diverse serotype O1 or O2 strains: primers wzm5′S2 and hisl3′AS2, and alternative longer versions (wzm5′S3 and hisl3′AS3) with higher Tₘ, are shown in Table 3. Using these primers, the 8.2 kb v1 (SEQ ID NO: 13) and 11.1 kb v2 (SEQ ID NO: 14) gene fragments (responsible for biosynthesis of respective galactans I and III) were PCR amplified from Klebsiella genomic DNA using a long PCR kit (Roche) and gel purified. To facilitate subcloning of these fragments, an oligonucleotide adaptor linker was designed to modify the polylinker cloning site of the pBAD33 vector. The double stranded adaptor contained the following features: a unique internal PmeI site cloning site; flanking 5′ and 3′ sequences homologous to the corresponding wzm and his/termini of v1 or v2 operon fragments; and single stranded ends compatible with pBAD33 vector linearized by SacI and HindIII restriction enzyme digestion. Sense and antisense adaptor primers were annealed and ligated into SacI/HindIII digested pBAD33 with T4 DNA ligase. The pBAD33 plasmid vector has a low-to-medium copy p15a replicon which can co-exist with CoIE1 replicons (medium or high copy number variants) for dual plasmid coexpression studies. After PmeI digestion, the v1 and v2 operon fragments were cloned into the modified acceptor vector using the high fidelity Gibson reaction enzyme mix according to kit instructions (Hifi builder, NEB). Resulting plasmids are listed in Table 4. A second higher copy CoIE1 replicon pBAD18 vector was similarly modified for v1 and v2 operon cloning using analogous adaptor primers compatible with vector NheI and HindIII sites. The pBAD18 and pBAD33 plasmid vectors contain the arabinose inducible promoter and express the AraC repressor and are described in Guzman L M, et al. Journal of bacteriology 1995; 177:4121-30. Plasmid transformants were selected on LB agar supplemented with chloramphenicol (30 mg/mL).

The unlinked genetic locus and WbbY and WbbZ enzymes responsible for synthesis of the immunodominant galactan II was identified originally by transposon mutagenesis (Hsieh P-F, et al. Frontiers in microbiology 2014; 5:608). The WbbY enzyme was later shown in vitro to work in concert with galactan I biosynthetic enzymes to add galactan II to the non-reducing end of galactan I to generate the chimeric galactan II-I (O1v1) O-antigen (Kelly S D, et al. J Biol Chem 2019; 294:10863-76). Formation of the galactan II-III (O1v2) O-antigen presumably forms by an analogous capping reaction in which galactan II is transferred to the galactan III. Using conserved primers flanking wbbyz genes of Klebsiella serotype O1 strains we amplified and cloned the corresponding gene fragments into a high copy number CoIE1 Topo vector (Invitrogen) (Table 2, Table 3, and Table 4). Plasmid transformants were selected on LB agar supplemented with Kanamycin (25 mg/mL).

TABLE 3
Oligonucleotide Primers

| Name | Sequence | Comments |
|---|---|---|
| wzm5′S2 | ATGAGTATAAAGATGAAGTACAATTTAGGGTAT | v1/v2 operon |
| | (SEQ ID NO: 16) | PCR |
| his13′AS2 | GAAGTGATTGATAATTTAAGAGCACGGCAT | v1/v2 operon |
| | (SEQ ID NO: 17) | PCR |
| wzm5′S3 | ATGAGTATAAAGATGAAGTACAATTTAGGGTAT | Longer wzm5′S2 |
| | TTATTTGATTTACTTGTTGT (SEQ ID NO: | |
| | 18) | |
| hisl3′AS3 | GGAAGTGATTGATAATTTAAGAGCACGGCATAG | Longer hisl3′AS2 |
| | G (SEQ ID NO: 19) | |
| pBAD33_O1O2 | CAACATAGGAGGAAATTATATGAGTATAAAGAT | pBAD33 Pmel |
| S | GAAGTACAATTTAGGGGTTTAAACCCTATGCCG | cloning adaptor |
| | TGCTCTTAAATTATCAATCACA (SEQ ID | S |
| | NO: 20) | |
| pBAD33_O1O2 | AGCTTGTGATTGATAATTTAAGAGCACGGCATA | pBAD33 Pmel |
| AS | GGGTTTAAACCCCTAAATTGTACTTCATCTTTA | cloning adaptor |
| | TACTCATATAATTTCCTCCTATGTTGAGCT | AS |
| | (SEQ ID NO: 21) | |
| pBAD18_O1O2 | CTAGCAACATAGGAGGAAATTATATGAGTATAA | pBAD18 Pmel |
| S | AGATGAAGTACAATTTAGGGGTTTAAACCCTAT | cloning adaptor |
| | GCCGTGCTCTTAAATTATCAATCACA (SEQ | S |
| | ID NO: 22) | |
| pBAD18_O1O2 | AGCTTGTGATTGATAATTTAAGAGCACGGCATA | pBAD18 Pmel |
| AS | GGGTTTAAACCCCTAAATTGTACTTCATCTTTA | cloning adaptor |
| | TACTCATATAATTTCCTCCTATGTTG (SEQ | AS |
| | ID NO: 23) | |
| wbbZY PCR | TGATTTAGCACTGCACTGAATTTGGG (SEQ | wbbzy PCR |
| S1 | ID NO: 24) | |
| wbbZY PCR | TATAGGCGTGCGAATGAATAGTCACCT (SEQ | wbbzy PCR |
| AS1 | ID NO: 25) | |

In Table 3 sense and antisense adaptor oligos used to modify pBAD vectors contain the unique PmeI cloning site (underlined) for introducing O1 and O2 v1 or v2 gene clusters. The start codon for the wzm gene and a 5′ ribosome binding site is highlighted in bold typeface with italics.

TABLE 4
Recombinant Plasmids

| | | Resis- | | | |
|---|---|---|---|---|---|
| | | tance | Klebsiella | Gene | |
| Name | Vector | marker | isolate | cluster | Antigen |
| pBAD33O1v1_ | pBAD33 | Cam | PFEKP0011 | 8.2 kb v1 | Galactan |
| 1-2 | | | | operon | I |
| pBAD33O1v2_ | pBAD33 | Cam | PFEKP0049 | 11.1 kb v2 | Galactan |
| 8-2 | | | | operon | III |
| pBAD33O1v2_ | pBAD33 | Cam | PFEKP0004 | 11.1 kb v2 | Galactan |
| 4-2 | | | | operon | III |
| pBAD33O2v1_ | pBAD33 | Cam | PFEKP0005 | 8.2 kb v1 | Galactan |
| 11-2 | | | | operon | I |
| pBAD33O2v2_ | pBAD33 | Cam | PFEKP0017 | 11.1 kb v2 | Galactan |
| 13-8 | | | | operon | III |
| pBAD18O2v1_ | pBAD18 | Cam | PFEKP0011 | 8.2 kb v1 | Galactan |
| 1-2 | | | | operon | I |
| pBAD18O2v1_ | pBAD18 | Cam | PFEKP0005 | 8.2 kb v1 | Galactan |
| 11-2 | | | | operon | I |
| pBAD18O2v2_ | pBAD18 | Cam | PFEKP0049 | 11.1 kb v2 | Galactan |
| 8-2 | | | | operon | III |
| pTopoZY_12 | Topo-II | Kan | PFEKP0011 | 3.4 kb | Galactan |
| | | | | wbbZY | II |
| pTopoZY_82 | Topo-II | Kan | PFEKP0049 | 3.4 kb | Galactan |
| | | | | wbbZY | II |

### D. Growth of Recombinant Strains and Small Scale O-Antigen Expression and Purification

For initial screening of recombinant E. coli plasmid transformants, 3 mL LB cultures were grown overnight with appropriate antibiotics and LPS extracted with phenol using a commercial kit (Bulldog-bio). Due to high basal expression from the pBAD arabinose promoter, arabinose inducer was not always necessary but in some cases was added to a level of 0.2%. Samples were run on an SDS-PAGE gradient gel under denaturing conditions (4-12%, Biorad). Carbohydrate was detected under UV light using a Pro-Q Emerald 300 staining kit (ThermoFisher).

A small shake-flask culture protocol was established to grow all four recombinant E. coli transformants in order to express and purify O-antigens which were further used for analytical characterization. To start, E. coli strains from frozen stocks were streaked on LB agar plates with 30 μg/ml chloramphenicol and/or 25 μg/ml kanamycin wherever appropriate (listed in Table 5) and incubated for 18 hours at 30° C. or 37° C. temperature (see Table 5). Then 3 mL of LB media (with listed antibiotics in Table 5) was inoculated with a single bacterial colony and grown overnight with shaking at the 30° C. or 37° C. temperature. Next 10 mL Apollon minimal media (with antibiotics) was inoculated with the LB seed culture (1:100 dilution) and grown over 24 hours at listed temperature (Table 5) with shaking at 250 rpm. Finally, after inoculation the bacteria were grown in 3×170 ml Apollon media (with listed antibiotics set forth in Table 4) in 500 mL baffled flask for 36-48 hours at 30° C. or 37° C. temperature. Bacteria was harvested by centrifugation (4000×g, 30 min) and the pellet was washed with water and resuspended in 300 ml of water and the pH was adjusted to 3.5 with glacial acetic acid followed by hydrolysis at 100° C. in a boiling water-bath. The suspension was cooled and then neutralized with 14% ammonium hydroxide. A solid-liquid separation was performed by centrifugation (9000×g, 25 min) and the supernatant was collected. Next, the crude O-antigen solution was flocculated using alum solution (2% w/v) and pH was adjusted to 3.2 using 1N sulfuric acid. After 1 h of incubation at room temperature the supernatant was collected after the centrifugation (12,000×g, 35 min, 15° C.) of the suspension. Further purification of O-antigen was accomplished by utilizing ultra-filtration/dia-filtration (UFDF) technique. Using a Ultracel 5 kD membrane in a Labscale Tangential Flow Filtration (TFF) system, first the O-antigen solution was reduced to ˜40 mL volume and then diafiltered first with 25 mM Citrate+0.1M NaCl buffer (20× diavolume) and then second diafiltration was performed with 25 mM Tris-HCl+25 mM NaCl buffer (20× diavolume). The UFDF retentate was then purified using anion-exchange membrane chromatography (with 25 mM Tris-HCl+25 mM NaCl elution buffer) and to the elute was added 4M ammonium chloride to make a final concentration of 2M. This mixture was purified by hydrophobic interaction chromatography (HIC) and the elute was collected. Final UFDF (5 kD Ultracel membrane, 30× diavolume of water) purification, extensive dialysis (3.5 kD dialysis cassette, 8×4 L water, room temp.), and final lyophilization yielded a significantly pure O-antigen in solid form.

### E. Carbohydrate Analytic Methods for Structural Confirmation

Purified O-antigen structure was characterized by 1D- and 2D-NMR recorded in a Bruker 600 MHz spectrometer equipped with TCI cryoprobe. The sample was deuterium exchanged and dissolved in deuterium oxide with 0.05% TSP (as internal standard). NMR data was analyzed using Bruker TopSpin 3.5 software. Recorded NMR chemical shifts (32 scans for proton and 4096 scans for carbon NMR) were compared with native Klebsiella O-antigen structures reported previously in the literature. Molar mass of the O-antigen was determined by SEC MALLS technique. Monosaccharide analysis of O-antigen was performed after hydrolyzing the sample with 2M trifluoroacetic acid at 95° C. for 4 h, drying the samples overnight in a speed-vac (room temperature), reconstituting in water followed by the HPAEC-PAD analysis (Dionex CarboPac PA1 column, 30° C.; Mobile phase: H2O and 200 mM NaOH) and peaks were compared against the standard monosaccharides (Fuc, Glc, Gal, GlcNAc, GalNAc, and Man).

### II. Results and Discussion

The carbohydrate repeat unit structures of the four predominant Klebsiella pneumoniae serotype O1 and O2 O-antigen subtypes O1v1, O1v2, O2v1, and O2v2 are shown in FIG. 1.

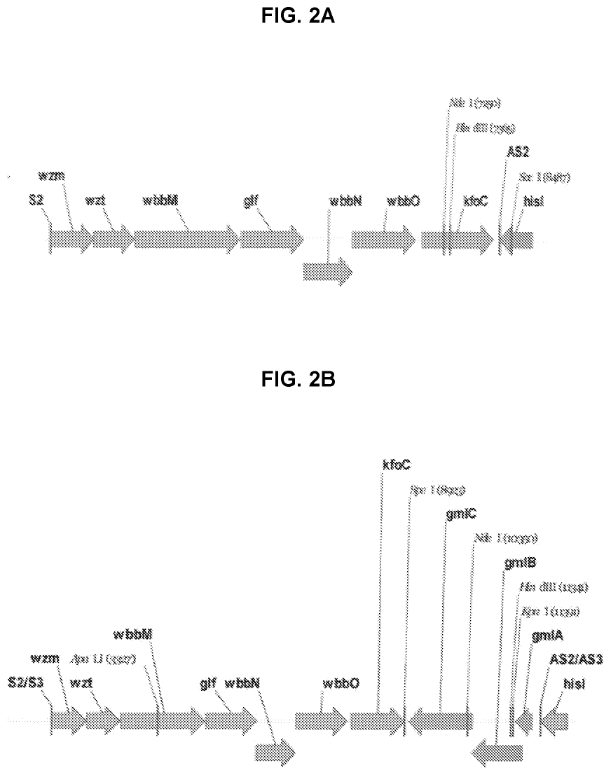

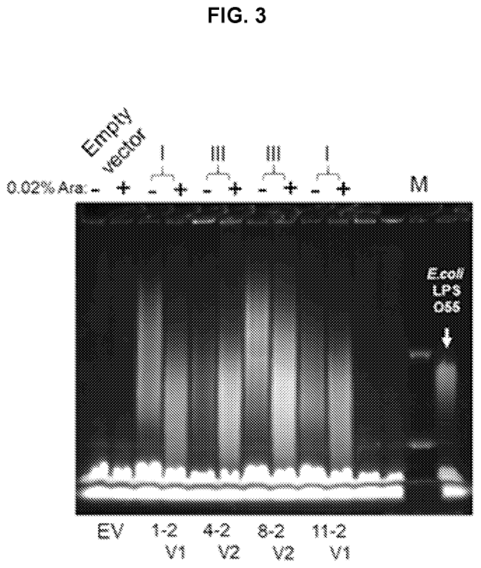

Sequencing of clinical strains allowed the identification of operons responsible for biosynthesis of galactan I (O2v1) and galactan III (O2v2) O-antigens. The organization of genes within v1 and v2 clusters obtained from representative strains is shown in FIG. 2.

Corresponding 8.2 kb and 11.1 kb fragments (DNA fragments containing respective v1 and v2 biosynthetic gene clusters) were PCR amplified and cloned into the p15a plasmid vector pBAD33 or the analogous CoIE1 replicon vector pBAD18. O-antigen deficient E. coli host strains were transformed with recombinant plasmid clones and expression of LPS O-antigens screened by SDS-PAGE with visualization via Emerald Green staining. Results of a representative experiment with pBAD33 subclones are shown in FIG. 3. While nothing is detected in the empty vector control, samples from v1 and v2 gene cluster subclones show a characteristic LPS profile. For some E. coli clones (clones 4-2 and 11-2), the presence of arabinose in the growth media improved expression, but in other cases good basal expression of LPS (clones 1-2 and 8-2) in the absence of arabinose was also observed. As the size distribution of clones 1-2 (Klebsiella PFEKP0011, v1 cluster) and 8-2 (Klebsiella PFEKP0049, v2 cluster) in the absence of arabinose indicated higher molecular mass than the others, these two bacterial transformants were selected for further analysis.

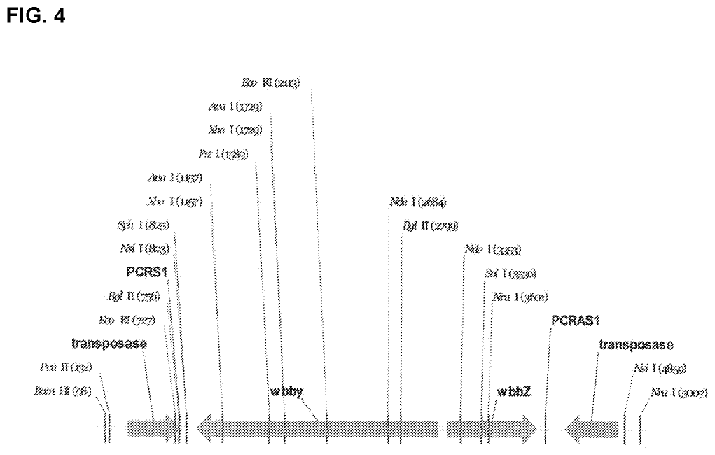

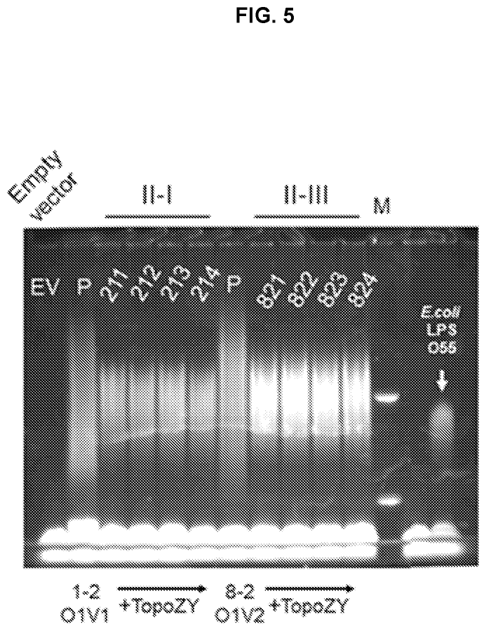

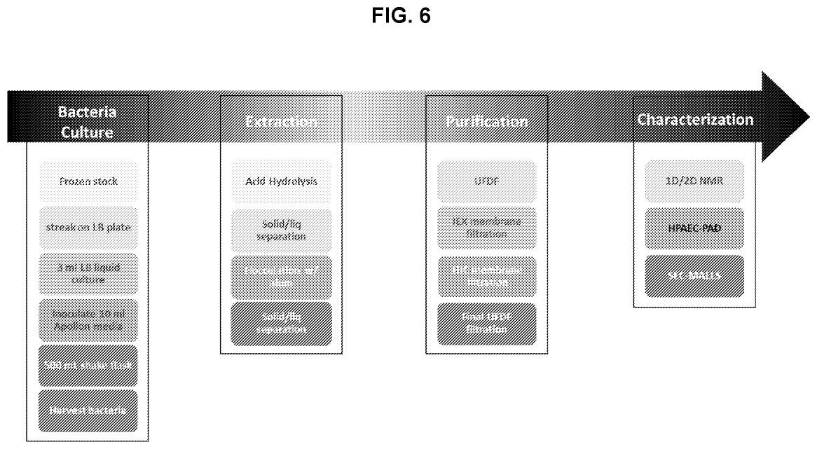

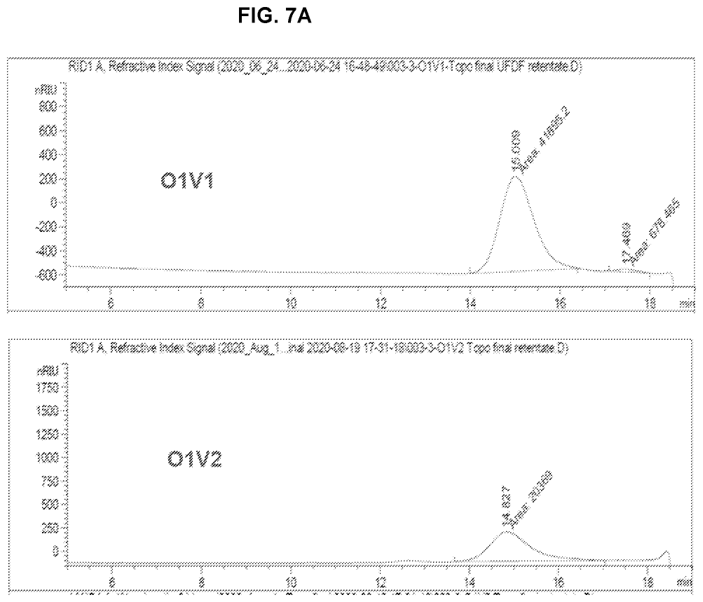

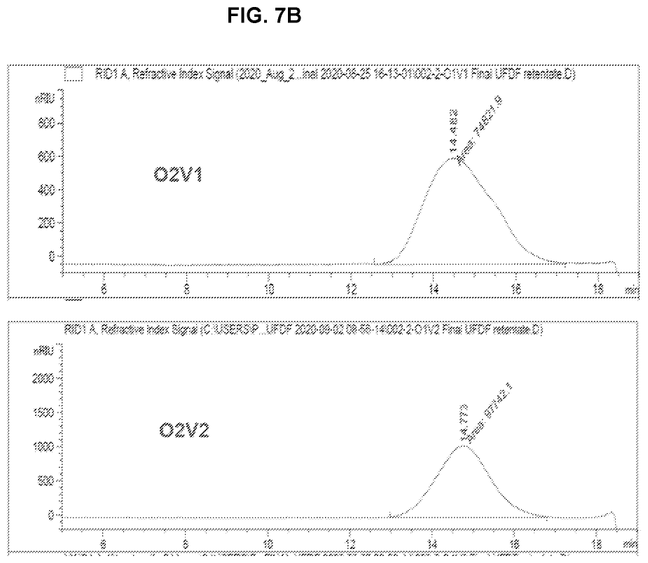

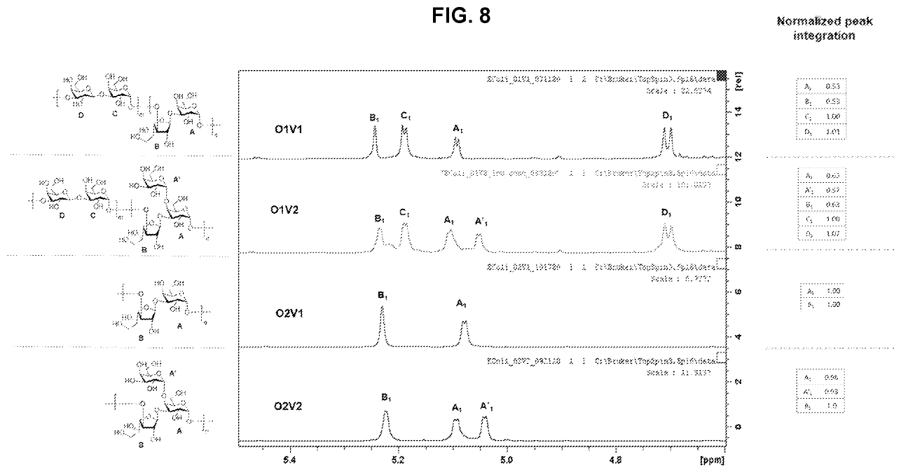

To generate chimeric galactans characteristic of the O1v1 and O1v2 subtypes, wbbY and wbbZ genes associated with galactan II production were PCR amplified from different Klebsiella clinical strains and cloned into the high-copy number CoIE1 Topo vector plasmid. The structure of the wbbyz locus deduced from WGS sequencing for representative Klebsiella strain PFEKP0011 is shown in FIG. 4. E. coli transformants harboring pBAD33 v1 or v2 clusters were transformed with a second compatible Topo wbbyz plasmid derived from the same Klebsiella strain. In the experiment shown in FIG. 5, LPS profiles from parental pBAD33 v1 or v2 single transformants (clones 1-2 or 8-2 in FIG. 3) are compared with corresponding double transformants harboring the additional wbbyz Topo plasmid. LPS extracted from the double transformants shows a distinct more uniform molecular mass staining profile compared with the parental single transformants. Representative double transformants were randomly selected for subsequent larger scale growth experiments.

The steps followed for small scale culture, purification, and characterization of O-antigens have been described in the Materials and Method section above. E. coli double transformants strains that express antigen O1v1 and O1v2 were grown in presence of 30 μg/ml Chloramphenicol and 25 μg/ml Kanamycin and incubated at 30° C. for 48 hours (see Table 5). On the other hand, single transformant E. coli strains were grown in presence of only 30 μg/ml Chloramphenicol and incubated at 37° C. for 36 hours. The OD values, culture media pH (after incubation), and final O-antigen yields are listed in Table 5.

TABLE 5
Growth of E. coli Recombinant Strains and Yields of Klebsiella O-antigens

| | | | | Incubation | | Culture | |
|---|---|---|---|---|---|---|---|
| | | | | time | | sup pH | O—Ag |
| Kleb | E. coli | Antibiotic | Incubation | (500 ml | Final | (after | Yield |
| O—Ag | transformant | Resistant | Temp | flask) | OD₆₀₀ | incubation) | (mg/L) |
| O1V1 | O1V1 1-2 | Cam^{R} + Kan^{R} | 30° C. | 48 h | 6.96 | 5.63 | 16 |
| | pBAD33 + | | | | | | |
| | Topo wzzby | | | | | | |
| O1V2 | O1V2 8-2 | Cam^{R} + Kan^{R} | 30° C. | 48 h | 7.11 | 5.12 | ~3 |
| | pBAD33 + | | | | | | |
| | Topo wzzby | | | | | | |
| O2V1 | O1V1 1-2 | Cam^{R} | 37° C. | 36 h | 5.90 | 5.11 | 14 |
| | pBAD33 | | | | | | |
| O2V2 | O1V2 8-2 | Cam^{R} | 37° C. | 36 h | 7.98 | 5.77 | 18 |
| | pBAD33 | | | | | | |

The surface O-antigen polysaccharide was extracted by acid hydrolysis and then purified as described in the Materials and Method section. During the purification of the O-antigen the purity and loss of sample was checked by HPLC-SEC analysis with RI detection after each step. For this, the sample was run through a size-exclusion column and monitored by UV (214 nm) and refractive index (RI).

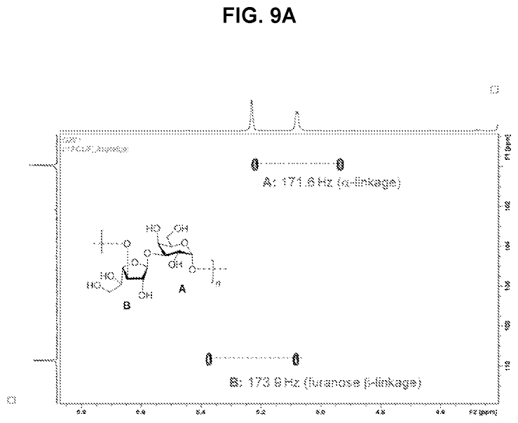

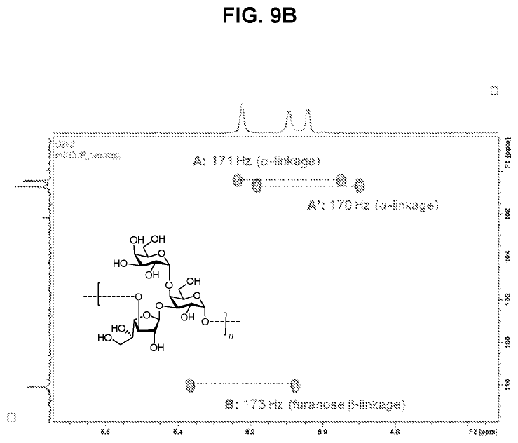

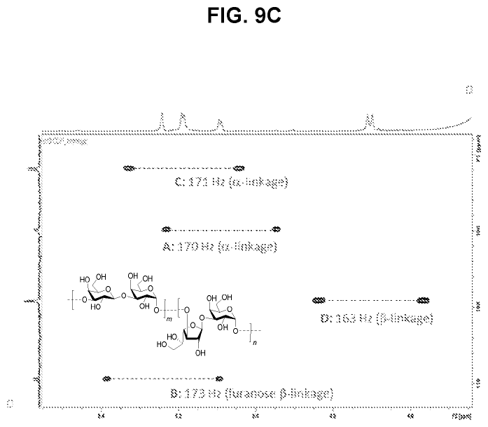

All the proton and carbon NMR signals were annotated by utilizing 1H- and 13C-NMR, 2D NMR such as COSY, HSQC, and HMBC. Due to low yield the acquisition of 2D NMR of O1V2 was not accomplished. However, comparing the NMR signals to the other antigen subtypes and the reported literature value (Table 6), we are confident about the peak annotation, which reveals the presence of Galactan I and Galactan III repeating unit. For the rest of the O-antigens, the linkage between the Galactose units was confirmed by overlaying HSQC and HMBC spectra. To understand the linkage stereochemistry, couple'd HSQC experiment was performed and the alpha- or beta-linkages were confirmed based on the measured proton-carbon coupling constants. The coupling constant values are indicated in the FIG. 9 below.

To validate the recombinant Klebsiella O-antigen structures expressed in E. coli, the NMR chemical shifts were compared to the native Klebsiella O-antigen structures reported in the literature (Vinogradov E, et al. J Biol Chem 2002; 277:25070-81). The chemical shift values are listed in Table 6 below.

TABLE 6
1H and 13C NMR Chemical Shift Comparison Between Reported and Expressed O-antigens

| | O1V1 | | | | | O2V1 | | | | | O2V2 | | | |
|---|---|---|---|---|---|---|---|---|---|---|---|---|---|---|
| | 1H (ppm) | | 13C (ppm) | | | 1H (ppm) | | 13C (ppm) | | | 1H (ppm) | | 13C (ppm) | |
| | Lit | Expmnt | Lit | Expmnt | | Lit | Expmnt | Lit | Expmnt | | Lit | Expmnt | Lit | Expmnt |
| A1 | 5.06 | 5.09 | 100.4 | 100.4 | A1 | 5.05 | 5.07 | 100.4 | 100.4 | A1 | 5.09 | 5.09 | 101.3 | 101.2 |
| A2 | 3.94 | 3.95 | 68.1 | 68.2 | A2 | 3.92 | 3.94 | 68.1 | 68.2 | A2 | 4.08 | 4.09 | 69.1 | 69 |
| A3 | 3.91 | 3.91 | 78 | 78 | A3 | 3.91 | 3.92 | 78 | 77.9 | A3 | 3.94 | 3.93 | 78.1 | 78.2 |
| A4 | 4.13 | 4.14 | 70.2 | 70.2 | A4 | 4.12 | 4.14 | 70.2 | 70.2 | A4 | 4.19 | 4.19 | 79.5 | 79.4 |
| A5 | 4.12 | 4.13 | 72.2 | 72.2 | A5 | 4.11 | 4.11 | 72.2 | 72.2 | A5 | 4.15 | 4.14 | 73.6 | 73.6 |
| B1 | 5.21 | 5.24 | 110.2 | 110.2 | A6 | 3.75 | 3.75 | 62.1 | 62.1 | A6a | 3.84 | 3.89 | 61.7 | 61.8 |
| B2 | 4.39 | 4.4 | 80.6 | 80.6 | B1 | 5.19 | 5.23 | 110.2 | 110.2 | A6b | 3.89 | — | — | — |
| B3 | 4.06 | 4.08 | 85.4 | 85.4 | B2 | 4.38 | 4.4 | 80.6 | 80.7 | B1 | 5.22 | 5.22 | 110.9 | 110.9 |
| B4 | 4.24 | 4.27 | 82.8 | 83 | B3 | 4.06 | 4.08 | 85.4 | 85.4 | B2 | 4.33 | 4.33 | 81.8 | 81.8 |
| B5 | 3.86 | 3.87 | 71.7 | 71.7 | B4 | 4.24 | 4.26 | 82.8 | 83 | B3 | 4.08 | 4.08 | 85.9 | 85.9 |
| C1 | 5.16 | 5.19 | 96.2 | 96.4 | B5 | 3.85 | 3.86 | 71.7 | 71.8 | B4 | 4.29 | 4.28 | 81.3 | 81.5 |
| C2 | 4.04 | 4.08 | 68.2 | 68.2 | B6 | 3.69 | 3.69 | 63.7 | 63.8 | B5 | 3.86 | 3.86 | 71.6 | 71.7 |
| C3 | 4.13 | 4.14 | 79.9 | 80 | | | | | | B6 | 3.69 | 3.69 | 64.2 | 64.2 |
| C4 | 4.26 | 4.26 | 70 | 70 | | | | | | A′1 | 5.03 | 5.04 | 101.6 | 101.5 |
| D1 | 4.67 | 4.7 | 105 | 105 | | | | | | A′2 | 3.83 | 3.84 | 70.3 | 70.4 |
| D2 | 3.74 | 3.78 | 70.5 | 70.7 | | | | | | A′3 | 3.91 | 3.9 | 70.5 | 70.6 |
| D3 | 3.78 | 3.77 | 78.1 | 78.4 | | | | | | A′4 | 4.06 | 4.06 | 70.1 | 70.3 |
| D4 | 4.17 | 4.12 | 65.7 | 66 | | | | | | A′5 | 4.2 | 4.19 | 72 | 72 |
| | | | | | | | | | | A′6a | 3.78 | 3.79 | 61.6 | 61.7 |
| | | | | | | | | | | A′6b | 3.81 | — | — | — |

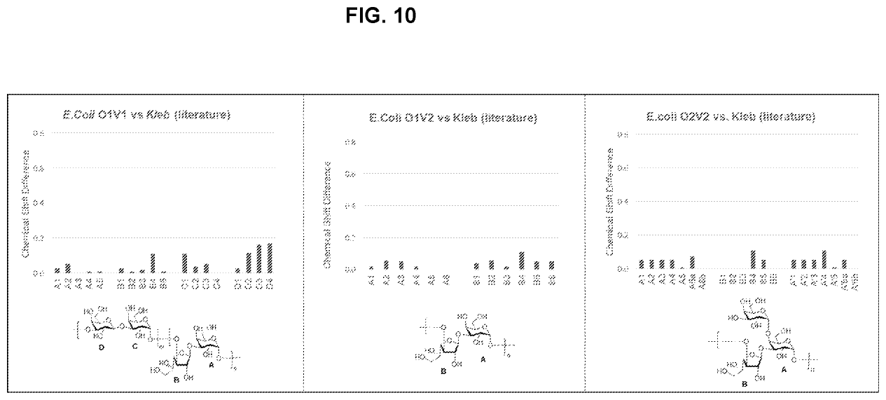

The CSD values were calculated for all the individual protons and carbons and plotted against them in the following chart (FIG. 10). No CSD value was obtained above 0.2, which indicates that the experimentally obtained recombinant Klebsiella O-antigen structures are in well accordance to the reported O-antigen structures expressed in native Klebsiella strains.

The proton NMR peak integration value was used to predict the number of Galactan repeating unit (RU) present in each polysaccharide. The ¹HNMR signal from the core region that appears at 05.45 ppm, was used to calculate the number of RU. The NMR-predicted values are listed in the following table (Table 7). Recombinantly expressed O-antigens were subjected to 2M TFA mediated hydrolysis at 100° C. and digested sample was analyzed by HPAEC-PAD technique. All the samples showed a preponderance of galactose monosaccharide units, a composition consistent with Klebsiella O1 and O2 O-polysaccharides. The intact O-antigens were also subjected to SEC-MALLS analysis to determine the molar mass of the polysaccharides. The molar mass obtained from the SEC MALLS study was compared with the calculated mass based on the NMR-predicted RU numbers (obtained by comparing proton peak integration values of anomeric proton and the core signal at 05.45 ppm). The predicted mass matches closely with the experimentally obtained molar mass of the O1V1 and O2V2.

TABLE 7
SEC-MALLS Data Confirms the
RU Molar Mass Predicted by NMR

| | | | | | Native |
|---|---|---|---|---|---|
| | | | | | O- |
| | | | | | antigen |
| | | | | Molar | molar |
| | Repeating | Predicted | Estimated | mass | mass |
| Klebsiella | Unit | number | molar | (SEC- | (from |
| O-antigen | (RU) | of RU | mass | MALLS) | EBPD) |
| O1V1 | Galactan II | Galactan | ~14.6 kDa | 15,920 Da | 13,000 Da |
| | + | II: 27 | | | |
| | Galactan I | Galactan | | | |
| | | I: 14 | | | |
| O2V1 | Galactan I | 38 | ~14 kDa | — | 10,960 Da |
| O2V2 | Galactan III | 55 | ~29 kDa | 28,230 Da | 12-58 kDa |

### III. Conclusion

Proof of concept for the expression of Klebsiella pneumoniae serotype O1 and O2 O-antigens in E. coli was established at exploratory shake-flask scale using a plasmid-based platform. Three biosynthetic gene clusters were cloned into plasmids and were capable of generating the desired individual or chimeric combinations of the three galactan components that comprise the two major O-antigen subtypes: O2v1 (galactan I); O2v2 (galactan III); O1v1 (galactan II-I chimera); and O1v2 (galactan II-III chimera). Analysis of the recombinant O-antigens extracted and purified at small scale confirm that they match the repeat unit structures of the corresponding native Klebsiella pneumoniae O-antigens. A minor difference between recombinant and native O-antigens is the presence in the E. coli material of terminal oligosaccharides at the reducing end due to differences in the placement of acid-labile Kdo sugars within the LPS oligosaccharide core. In case of Klebsiella, acid hydrolysis has the potential to cleave the core more completely from the O-antigen because of the presence of a Kdo unit towards the outer core (Vinogradov E, et al. J Biol Chem 2002; 277:25070-81). In contrast, the host E. coli K12 core has Kdo units only towards the reducing end of the inner core (Heinrichs D E, et al. Molecular microbiology 1998; 30:221-32). These residual E. coli core oligosaccharides are not expected to contribute to the functional immunogenicity of derived glycoconjugate antigens, as core-specific antibody binding epitopes are not exposed on the surface of E. coli O-antigen expressing strains, as demonstrated in flow cytometry experiments (data not shown).

For scalable bioprocessing it may be desirable to stably integrate these gene clusters into the E. coli host chromosome. This may be accomplished by site specific genome recombination or by standard homologous recombination methods (Haldimann A, Wanner B L. Journal of bacteriology 2001; 183:6384-93; Lynn Thomason D L C, Mikail Bubunenko, Nina Costantino, Helen Wilson S D, and Amos Oppenheim. Recombineering: genetic engineering in bacteria using homologous recombination. In: F. M. Ausubel R B, R. E. Kingston, D. D. Moore, J. G. Seidman, J. A. Smith, K. Struhl, ed. Current Protocols in Molecular Biology. Vol. 1.16.1-1.16.24. Hoboken, N.J.: John Wiley & Sons, Inc, 2007: pp. 1-21).

### SEQUENCES

TABLE 8
O2v1 gene cluster (K.pn. O2 O-Ag Galactan I biosynthetic
gene cluster [FIG. 2] (8.2kb v1 operon)

| vector | pBAD33 (p15a replicon) or pBAD18 (ColE1 replicon) | |
|---|---|---|
| SEQ ID | Protein name | |
| NO: | (gene) | Sequence |
| 1 | i) Transport | >tr\|070068\|O70068_KLEPN Transport permease |
| | permease protein | protein OS = Klebsiella pneumoniae OX = 573 |
| | (wzm) | GN = wzm PE = 3 SV = 1 |
| | | MSIKMKYNLGYLFDLLVVITNKDLKVRYKSSMLGYLWSVANPLLFAMI |
| | | YYFIFKLVMRVQIPNYTVFLITGLFPWQWFASSATNSLFSFIANAQII |
| | | KKTVFPRSVIPLSNVMMEGLHFLCTIPVIVVFLFVYGMTPSLSWVWGI |
| | | PLIAIGQVIFTFGVSIIFSTLNLFFRDLERFVSLGIMLMFYCTPILYA |
| | | SDMIPEKFSWIITYNPLASMILSWRDLFMNGTLNYEYISILYFTGIIL |
| | | TVVGLSIFNKLKYRFAEIL |
| 2 | ii) ABC | >tr\|A0A0S3TG60\|A0A0S3TG60_KLEPN ABC transporter, |
| | transporter, | ATP-binding component OS = Klebsiella pneumoniae |
| | ATP-binding | OX = 573 GN = wzt PE = 4 SV = 1 |
| | component (wzt) | MHPVINFSHVTKEYPLYHHIGSGIKDLIFHPKRAFQLLKGRKYLAIED |
| | | VSFTVGKGEAVALIGRNGAGKSTSLGLVAGVIKPTKGTVTTEGRVASM |
| | | LELGGGFHPELTGRENIYLNATLLGLRRKEVQQRMERIIEFSELGEFI |
| | | DEPIRVYSSGMLAKLGFSVISQVEPDILIIDEVLAVGDIAFQAKCIQT |
| | | IRDFKKRGVTILFVSHNMSDVEKICDRVIWIENHRLREVGSAERIIEL |
| | | YKQAMA |
| 3 | iii) Glycosyl- | >tr\|M5B1W3\|M5B1W3_KLEPN Glycosyltransferase |
| | transferase | OS = Klebsiella pneumoniae OX = 573 GN = wbbM |
| | (wbbM) | PE = 4 SV = 1 |
| | | MNNSVKIYTSHHKPSAFLNAAIIKPLHVGKANSCNEIGCPGDDTGDNI |
| | | SFKNPFYCELTAHYWVWKNEELADYVGFMHYRRHLNFSEKQTFSEDTW |
| | | GVVNHPCIDEEYEKIFGLNEETIQRCVEGIDILLPKKWSVTAAGSKNN |
| | | YDHYERGEYLHIRDYQAAIAIVEKLYPEYSAAIKTFNDASDGYYTNMF |
| | | VMRKDIFVDYSEWLFSILDNLEDAISMNNYNAQEKRVIGHIAERLENI |
| | | YIIKLQQDGELKVKELQRTFVSNETFNGALNPVFDSAVPVVISFDDNY |
| | | AVSGGALINSIVRHADKNKNYDIVVLENKVSYLNKTRLVNLTSAHPNI |
| | | SLRFFDVNAFTEINGVHTRAHFSASTYARLFIPQLFRRYDKVVFIDSD |
| | | TVVKADLGELLDVPLGNNLVAAVKDIVMEGFVKFSAMSASDDGVMPAG |
| | | EYLQKTLNMNNPDEYFQAGIIVFNVKQMVEENTFAELMRVLKAKKYWF |
| | | LDQDIMNKVFYSRVTFLPLEWNVYHGNGNTDDFFPNLKFATYMKFLAA |
| | | RKKPKMIHYAGENKPWNTEKVDFYDDFIENIANTPWEMEIYKRQMSLA |
| | | ASIGLTHSEPQQQILFQTKIKNVLMPYVNKYAPIGTPRRNMMTKYYYK |
| | | VRRAILG |
| 4 | iv) UDP-galacto- | >sp\|Q48485\|GLF1_KLEPN UDP-galactopyranose mutase |
| | pyranose mutase | OS = Klebsiella pneumoniae OX = 573 GN = rfbD |
| | (glf) | PE = 1 SV = 1 |
| | | MKSKKILIVGAGFSGAVIGRQLAEKGHQVHIIDQRDHIGGNSYDARDA |
| | | ETNVMVHVYGPHIFHTDNETVWNYVNKHAEMMPYVNRVKATVNGQVFS |
| | | LPINLHTINQFFSKTCSPDEARALIAEKGDSTIADPQTFEEQALRFIG |
| | | KELYEAFFKGYTIKQWGMQPSELPASILKRLPVRFNYDDNYFNHKFQG |
| | | MPKCGYTQMIKSILNHENIKVDLQREFIVEERTHYDHVFYSGPLDAFY |
| | | GYQYGRLGYRTLDFKKFTYQGDYQGCAVMNYCSVDVPYTRITEHKYFS |
| | | PWEQHDGSVCYKEYSRACEENDIPYYPIRQMGEMALLEKYLSLAENET |
| | | NITFVGRLGTYRYLDMDVTIAEALKTAEVYLNSLTENQPMPVFTVSVR |
| 5 | v) Galactosyl- | >tr\|Q48486\|Q48486_KLEPN WbbN protein OS = |
| | transferase | Klebsiella pneumoniae OX = 573 GN = wbbN |
| | (wbbN) | PE = 4 SV = 1 |
| | | MKYTALIVTFNRLGKLKKTVEETLKLEFTNIVIVNNGSTDGTQAWLSS |
| | | IVDTRVIVLTLTENTGGAGGFKTGSQYICEQLASDWVFFYDDDAYPYP |
| | | DTLKSFSQLDKQGCRVFSGLVKDPQGKPCPMNMPFSRVPTSLGDTVRY |
| | | LRYPGEFIPAANRSMFVQTVSFVGMVIHRDLLTTSLDHIHEQLFIYFD |
| | | DLYFGYQLSLAGEKIMYSPELLFYHDVSIQGKLIAPEWKVYYLCRNLI |
| | | LSKKIFQKNGVYSNSAIAIRILKYILILPWQRQKYSYMKFILRGISHG |
| | | IKGISGKYH |
| 6 | vi) Galactosyl- | >tr\|Q48483\|Q48483_KLEPN Galactosyltransferase |
| | transferase | OS = Klebsiella pneumoniae OX = 573 GN = wbbO |
| | (wbbO) | PE = 4 SV = 1 |
| | | MRKLCYFINSDWYFDLHWIDRAIASRDAGYEIHIISHFIDDNIINKFK |
| | | TFGFICHNVTLDAQSFNALVFFRTYHDVQKIIKNIKPDLLHCITIKPC |
| | | LIGGVLAKKFNLPVIVSFVGLGRVFSSDSMPLKLLRQFTIAAYKYIAS |
| | | NKRCIFMFEHDRDRKKLAKLVGLEEQQTIVIDGAGINPEIYKYSLEQN |
| | | HDVPVVLFASRMLWSKGLGDLIEAKKILRSKNIHFTLNVAGILVENDK |
| | | DAISLQVIENWHQQGLINWLGRSNNVCDLIEQSNIVALPSVYSEGVPR |
| | | ILLEASSVGRACIAYDVGGCDSLIIDNDNGIIVKSNSPEELADKLAFL |
| | | LSNPKARVEMGIKGRKRIQDKFSSGMIISKTLKTYHDVVEG |
| 7 | vii) FGlycosyl- | >tr\|A0A193SF76\|A0A193SF76_KLEPN FGlycosyl |
| | transferase | transferase family 2 OS = Klebsiella pneumoniae |
| | family 2 (kfoC) | OX = 573 GN = kfoC_1 PE = 4 SV = 1 |
| | | MSERSSSALVSVVIPVHDAAEYISDTLSSILSQSLQDIEVIIIDDNSA |
| | | DDTLKLLQSFAANDSRIRLLNNSQNIGAGASRNMGLKIASGEYIIFLD |
| | | DDDYADANMLKRMYDHAALLQADVVICRCQSLDLQTHSYAPMPWSVRV |
| | | DLLPQKELFSSDEITHNFFDAFIWWPWDKLFRRQAILDTGLQFQDLRT |
| | | TNDLFFVSAFMLLTKRMAFLDEILISHSINRSGSLSVTREKSWHCALD |
| | | ALRALYSFIDSKHLLPSRGRDFNNYAVTFLEWNLNTISGPAFDSLFTA |
| | | SREFIASLDIDESDFYDDFIKAAHYRLIRLTPEEYLFSLKDRVLHELE |
| | | SSNLSTEKLQASIASQDQVLKAREEEIDELRASVAQKKERIDRLMERN |
| | | AYLETEYQKQQDQLTKLQNELNNAAQRYSALISSLSWKVTRPLRLIKA |
| | | LIVKKM |

TABLE 9
O2v2 gene cluster (K.pn. O2 O-Ag Galactan III biosynthetic
gene cluster [FIG. 2] (11.1kb v2 operon)

| SEQ ID | Protein name | |
|---|---|---|
| NO: | (gene) | Sequence |
| vector | pBAD33 (p15a replicon) or pBAD18 (ColE1 replicon) | |
| 1 | (wzm) | same as O2v1 |
| 2 | (wzt) | MHPVINFSHVTKEYPLYHHIGSGIKDLIFHPKRAFQLLKGRKYLAIEDVSFTV |
| | | GKGEAVALIGRNGAGKSTSLGLVAGVIKPTKGTVTTEGRVASMLELGGGFHPE |
| | | LTGRENIYLNATLLGLRRKEVQQRMERIIEFSELGEFIDEPIRVYSSGMLAKL |
| | | GFSVISQVEPDILIIDEVLAVGDIAFQAKCIKTIRDFKKRGVTILFVSHNMSD |
| | | VEKICDRVIWIENHRLREVGSAERIIELYKQAMA |
| 3 | (wbbM) | VGNIMNNSVKIYTSHHKPSAFLNAAIIKPLHVGKANSCNEIGCPGDDTGDNIS |
| | | FKNPFYCELTAHYWVWKNEELADYVGFMHYRRHLNFSEKQTFSEDTWGVVNHP |
| | | CIDEEYEKIFGLNEETIQRCVEGIDILLPKKWSVTAAGSKNNYDHYERGEYLH |
| | | IRDYQAAIAIVEKLYPEYSAAIKTFNDASDGYYTNMFVMRKDIFVDYSEWLFS |
| | | ILDNLEDAISMNNYNAQEKRVIGHIAERLFNIYIIKLQQDGELKVKELQRTFV |
| | | SNETFNGALNPVFDSAVPVVISFDDNYAVSGGALINSIVRHADKNKNYDIVVL |
| | | ENKVSYLNKTRLVNLTSAHPNISLRFFDVNAFTEINGVHTRAHFSASTYARLF |
| | | IPQLFRRYDKVVFIDSDTVVKADLGELLDVPLGNNLVAAVKDIVMEGFVKFSA |
| | | MSASDDGVMPAGEYLQKTLNMNNPDEYFQAGIIVFNVKQMVEENTFAELMRVL |
| | | KAKKYWFLDQDIMNKVFYSRVTFLPLEWNVYHGNGNTDDFFPNLKFATYMKFL |
| | | AARKKPKMIHYAGENKPWNTEKVDFYDDFIENIANTPWEMEIYKRQMSLAASI |
| | | GLTHSEPQQQILFQTKIKNVLMPYVNKYAPIGTPRRNMMTKYYYKVRRAILG |
| 4 | (glf) | MKSKKILIVGAGFSGAVIGRQLAEKGHQVHIIDQRDHIGGNSYDARDAETNVM |
| | | VHVYGPHIFHTDNETVWNYVNKHAEMMPYVNRVKATVNGQVFSLPINLHTINQ |
| | | FFSKTCSPDEARALIAEKGDSTIADPQTFEEQALRFIGKELYEAFFKGYTIKQ |
| | | WGMQPSELPASILKRLPVRFNYDDNYFNHKFQGMPKCGYTQMIKSILNHENIK |
| | | VDLQREFIVEERTHYDHVFYSGPLDAFYGYQYGRLGYRTLDFKKFTYQGDYQG |
| | | CAVMNYCSVDVPYTRITEHKYFSPWEQHDGSVCYKEYSRACEENDIPYYPIRQ |
| | | MGEMALLEKYLSLAENETNITFVGRLGTYRYLDMDVTIAEALKTAEVYLNSLT |
| | | ENQPMPVFTVSVR |
| 5 | (wbbN) | MKYTALIVTFNRLGKLKKTVEETLKLEFTNIVIVNNGSTDGTQAWLSSIVDTR |
| | | VIVLTLTKNTGGAGGFKTGSQYICEQLASDWVFFYDDDAYPYPDTLKSFSQLD |
| | | KQGCRVFSGLVKDPQGKPCPMNMPFSRVPTSLGDTVRYLRYPGEFIPAANRSM |
| | | FVQTVSFVGMVIHRDLLATSLDHIHEQLFIYFDDLYFGYQLSLAGEKIMYSPE |
| | | LLFYHDVSIQGKLIAPEWKVYYLCRNLILSKKIFQKNAVYSNSAIAIRILKYI |
| | | LILPWQRQKYSYMKFILRGISHGIKGISGKYH |
| 6 | (wbbO) | MRKLCYFINSDWYFDLHWIDRAIASRDAGYEIHIISHFIDDNIINKFKTFGFI |
| | | CHNVTLDAQSFNALVFFRTYHDVQKIIKNIKPDLLHCITIKPCLIGGVLAKKE |
| | | NLPVIVSFVGLGRVFSSDSMPLKLLRQFTIAAYKYIASNKRCIFMFEHDRDRK |
| | | KLAKLVGLEEQQTIVIDGAGINPEIYKYSLEQDHDVPVVLFASRMLWSKGLGD |
| | | LIEAKKILRSKNIHFTLNVAGILVENDKDAISLQVIENWHQQGLINWLGRSNN |
| | | VCDLIEQSNIVALPSVYSEGVPRILLEASSVGRACIAYDVGGCDSLIIDNDNG |
| | | IIVKSNSPEELADKLAFLLSNPKARVEMGIKGRKRIQDKFSSVMIIDKTLQIY |
| | | HDVVR |
| 7 | (kfoC) | MAHEKSDIIVSVVIPVYNAEEYIADTLKNIVSQSLYEIEIIIINDHSSDNTLD |
| | | ILKEIASSDERIRIIDNAVNIGAGISRNIGLSEAKGEYIIFLDDDDYVDTNML |
| | | KHMSDCAELSGADIVVCRSRSFNLQSLQYAPMPDSIRKDLLPEKAVFSPGDIE |
| | | RDFFRAFIWWPWDKLFRREFIIQHSLSYQDLRTSNDLFFVCASMLSAEKVTIL |
| | | DEILITHTINRKTSLSSTRSVSYHCALDALVALRDFLFKNGMMQKRQRDFYNY |
| | | IVVFLEWHLNTLSGEAFNKLFQDVKLFISSFDINNEDFYDEFILSAYRRIADM |
| | | SAEEYLFSLKDRVINELENAQRNILTLQNEVEEIKQQLQQKDEMIASMNRENL |
| | | AIKADNKILENYNEELKTVQTKFLKLLSSKD |
| 8 | GmIC protein | MENNMQNLINPLAEGNKKNVYIFYFFLLMLTFSPVIFFSYAFSDDWSTLFDAI |
| | (gmIC) | TRNGSSFQWDVQSGRPVYAVFRYYGKMLINDISSFSYLRLFNILSLVVLSCFI |
| | | YNFIDSRKIFDNPVFKIIFPLLICLLPAFQVYASWATCFPFTISVLLAGISYN |
| | | KCFPHSKQRSSLPEKLASIVVLWVAFAIYQPTAITFLFFFMLDSCIKKESSLT |
| | | VKKVATCFIILVIGVAGSFIMSKVLPVWLYGESLSRAELTADIGGKMKWFINE |
| | | SLINAVNNYNIQPVKIYSWFSSFAILIGLYTIFVGKTGRWKTFIVITIGIGSY |
| | | APNLATKENWAAFRSLVALELIISTLFLIGINSLVSRISKQAFVWPLIALTIM |
| | | IIAQYNIINGFIIPQRSEIQALAAEITNKIPKNYTGKLMFDLTDPAYNAFTKT |
| | | QRYDEFGNISLAAPWALKGMAEEIRIMKGFNFKLSNNVIISETNRCIDDCMVI |
| | | KTSDAMRRSTINY |
| 9 | GmIB protein | >tr\|A0A2L0WT46\|A0A2L0WT46_KLEPN GmIB OS = Klebsiella |
| | (gmIB) | pneumoniae OX = 573 GN = gmIB PE = 4 SV = 1 |
| | | MTTSTDIKSTPSLAIVVPCYNEQEAFPFCLEKLSNVLNSLIARNKINNNSYLL |
| | | FVDDGSRDNTWAQIKDASTAYHYVRGIKLSRNKGHQIALMAGLRSVDTDVTIS |
| | | IDADLQDDVNCIEKMIDAYSQGYDIVYGVRGNRDSDTFFKRTTANAFYAIMSH |
| | | LGVNQTPNHADYRLLSNRALEALKQYKEQNIYLRGLVPLVGYPSIEVQYSREE |
| | | RIAGESKYPIKKMLALALEGITSLSVTPLRIIAMTGFITCIISTIAAIYALIQ |
| | | KTTGTTVEGWTSVMIAIFFLGGVQMLSLGIIGEYVGKIYIETKNRPKYFIDES |
| | | VGNDSNGK |
| 10 | GmIA protein | >tr\|A0A2L0WT49\|A0A2L0WT49_KLEPN GmIA OS = Klebsiella |
| | (gmIA) | pneumoniae OX = 573 GN = gmIA PE = 3 SV = 1 |
| | | MPSSGPLWQLMKYGLVGIVNTLITAVVIFLLMHLGLGIYLSNAMGYVVGIVFS |
| | | FIANTIFTFTQPISINRLIKFLCVCFICYVANIIVIKIFFVFMPEKIYSAQIL |
| | | GMFTYTITGFILNKFWAMK |

TABLE 10
O1v1 & O1v2 gene cluster (K.pn. O1 O-Ag Galactan II biosynthetic
gene cluster [FIG. 4] (3.4kb wbbZY fragment)

| SEQ ID | Protein name | |
|---|---|---|
| NO: | (gene) | Sequence |
| vector | Topo-II (ColE1 replicon) | |
| 11 | Glycosyl- | >tr\|A0A0K2QTR0\|A0A0K2QTR0_KLEPN Glycosyltransferase |
| | transferase | OS = Klebsiella pneumoniae OX = 573 GN = wbbY |
| | (wbbY) | PE = 4 SV = 1 |
| | | MKKILIMTPDIEGPVRNGGIGTAFTALATTLAKKGYDVDVLYTCGDYSESS |
| | | VSKFSDWSRIYSTFGINLLRTGLIKEINIDAPYFRRKSYSIYLWLKENNIY |
| | | DTVISCEWQADLYYTLLSKKNGTDFENTKFIVNTHSSTLWADEGNYQLPYD |
| | | QNHLELYYMEKMVVEMADEVVSPSQYLIDWMLSKHWNVPEERHVILNCEPF |
| | | QGFVTRDDVTVKINEKPASGVELVFFGRLETRKGLDIFLRALRKLSDEDKE |
| | | SISGVTFLGKNVTMGKTDSFTYIMNQTKNLGLAVNVISDYDRTNANEYIKR |
| | | KNVLVIIPSLVENSPYTVYECLINNVNFLASNVGGIPELIPQEHHAEVLFI |
| | | PTPVDLYGKIHYRLKNINIKPGLAESQDNIKEAWFVAVERKNNRAFKKIDE |
| | | ANSPLVSVCITHFERHHLLQQALASIKSQTYQNIEVILVDDGSTTEDSHRY |
| | | LNLIENDFNSRGWKIVRSSNNYLGAARNLAARHASGEYLMFMDDDNVAKPF |
| | | EVETFVTAALNSGADVLTTPSDLIFGEEFPSPFRKMTHCWLPLGPDLNIAS |
| | | FSNCFGDANALIRKEVFEKVGGFTEDYGLGHEDWEFFAKISLQGYKLQIVP |
| | | EPLFWYRVANSGMLLSGNKSKNNYRSFRPFMDENVKYNYAMGLIPSYLEKI |
| | | QELESEVNRLRSINGGHSVSNELQLLNNKVDGLISQQRDGWAHDRFNALYE |
| | | AIHVQGAKRGTSLVRRVARKVKSMLK |
| 12 | Exopoly- | >tr\|A0A0J4KNC3\|A0A0J4KNC3_KLEPN Exopolysaccharide |
| | saccharide | biosynthesis protein OS = Klebsiella pneumoniae |
| | biosynthesis | OX = 573 GN = wbbZ PE = 4 SV = 1 |
| | protein | MTNMKLKFDLLLKSYHLSHRFVYKANPGNAGDGVIASATYDFFERNALTYI |
| | (wbbZ) | PYRDGERYSSETDILIFGGGGNLIEGLYSEGHDFIQNNIGKFHKVIIMPST |
| | | IRGYSDLFINNIDKFVVFCRENITFDYIKSLNYEPNKNVFITDDMAFYLDL |
| | | NKYLSLKPIYKKQANCFRTDSESLTGDYKENNHDISLTWNGDYWDNEFLAR |
| | | NSTRCMINFLEEYKVVNTDRLHVAILASLLGKEVNFYPNSYYKNEAVYNYS |
| | | LFNRYPKTCFITAS |

TABLE 11

| SEQ | | |
|---|---|---|
| ID | | |
| NO: | Name | Sequence |
| 13 | 8.2kb v1 | ATGAGTATAAAGATGAAGTACAATTTAGGGTATTTATTTGATTTACTTGT |
| | operon | TGTGATAACAAATAAAGATCTAAAAGTGCGCTATAAGAGCAGCATGCTAG |
| | fragment | GCTATTTATGGTCAGTAGCAAATCCATTGCTTTTTGCCATGATTTATTAT |
| | (Gal I | TTTATATTTAAGCTGGTAATGAGAGTACAAATTCCAAATTATACAGTTTT |
| | biosynthetic | CCTCATTACCGGCTTGTTTCCGTGGCAATGGTTTGCCAGTTCGGCCACTA |
| | gene cluster) | ACTCATTATTTTCATTCATCGCTAACGCTCAAATTATCAAGAAGACAGTT |
| | | TTTCCCCGTTCCGTGATTCCGCTAAGTAATGTGATGATGGAAGGCTTGCA |
| | | TTTTCTTTGCACCATCCCGGTTATTGTTGTCTTTCTTTTTGTTTATGGCA |
| | | TGACGCCGTCCTTGTCCTGGGTTTGGGGTATACCTCTCATTGCTATTGGC |
| | | CAGGTGATTTTCACCTTTGGTGTTTCAATCATCTTTTCAACGCTGAACCT |
| | | GTTTTTCCGTGACCTGGAGCGCTTTGTCAGTCTGGGGATTATGCTGATGT |
| | | TTTATTGTACGCCGATTTTATATGCGTCTGATATGATTCCGGAAAAATTT |
| | | AGCTGGATAATTACCTACAATCCGCTAGCGAGTATGATTCTTAGTTGGCG |
| | | TGATTTATTCATGAATGGGACTCTTAATTATGAGTATATTTCTATACTCT |
| | | ATTTTACGGGAATCATTTTGACGGTTGTCGGTTTGTCTATTTTCAATAAA |
| | | TTAAAATATCGATTTGCAGAGATCTTGTAATGCACCCAGTTATTAACTTC |
| | | AGTCATGTTACAAAAGAGTATCCTCTGTACCATCATATTGGCTCAGGAAT |
| | | CAAAGATTTAATTTTCCATCCAAAACGCGCTTTTCAGTTGCTGAAGGGGC |
| | | GGAAATATTTAGCTATCGAAGACGTATCCTTTACAGTTGGCAAAGGTGAG |
| | | GCTGTTGCCCTGATTGGACGTAATGGGGCAGGAAAGAGTACCTCGCTTGG |
| | | CCTGGTTGCCGGCGTGATTAAGCCAACTAAGGGAACCGTCACCACTGAAG |
| | | GACGGGTGGCATCGATGCTTGAACTCGGCGGAGGCTTTCATCCTGAACTT |
| | | ACCGGGCGTGAGAATATTTACCTGAATGCTACTCTGCTGGGCCTTCGGCG |
| | | TAAAGAGGTCCAGCAACGTATGGAACGTATTATTGAATTTTCGGAACTGG |
| | | GAGAATTCATAGACGAGCCAATCAGAGTGTACTCAAGCGGAATGCTAGCT |
| | | AAGTTAGGTTTTTCGGTCATCAGTCAGGTTGAACCGGATATTTTAATTAT |
| | | TGATGAAGTTCTGGCAGTAGGTGATATCGCTTTTCAGGCAAAATGTATTC |
| | | AGACCATCAGAGATTTTAAGAAAAGAGGCGTGACAATATTGTTTGTTAGC |
| | | CACAATATGAGTGACGTTGAAAAAATCTGCGACAGAGTCATCTGGATCGA |
| | | AAATCATAGGCTCAGAGAAGTGGGGTCTGCAGAGCGAATCATTGAACTGT |
| | | ACAAGCAAGCAATGGCTTAATCAGTGGGTAATATAATGAACAATAGCGTT |
| | | AAAATCTATACCAGCCACCATAAGCCTAGTGCTTTTCTTAATGCTGCAAT |
| | | TATCAAACCTCTGCATGTCGGCAAAGCTAATTCTTGTAATGAAATTGGTT |
| | | GTCCAGGAGATGACACTGGCGATAATATTTCCTTTAAGAATCCGTTTTAT |
| | | TGCGAACTAACTGCGCATTATTGGGTTTGGAAAAACGAAGAGCTGGCAGA |
| | | CTATGTCGGTTTCATGCACTATCGCCGTCATCTTAATTTTTCCGAAAAAC |
| | | AAACTTTTTCTGAGGATACCTGGGGGGTCGTGAACCATCCATGCATTGAT |
| | | GAAGAATATGAGAAGATCTTTGGATTAAACGAAGAAACAATTCAACGGTG |
| | | TGTCGAAGGTATTGACATCTTGCTGCCCAAAAAATGGTCTGTCACTGCGG |
| | | CGGGAAGTAAAAATAATTACGATCACTATGAACGAGGTGAATACTTACAT |
| | | ATTCGTGATTATCAGGCTGCCATTGCCATCGTTGAAAAACTATATCCAGA |
| | | GTATAGCGCGGCAATAAAAACGTTTAATGATGCCAGTGATGGCTATTACA |
| | | CAAATATGTTTGTCATGCGCAAAGATATTTTTGTTGACTATTCTGAGTGG |
| | | CTCTTTTCCATTCTGGATAATCTCGAAGATGCTATCTCGATGAACAATTA |
| | | TAATGCTCAGGAAAAACGCGTTATTGGGCATATAGCAGAACGGCTGTTTA |
| | | ATATTTACATTATTAAGTTGCAACAAGATGGTGAGCTTAAGGTAAAAGAA |
| | | TTACAGCGTACTTTTGTCAGCAATGAAACATTCAATGGTGCACTGAATCC |
| | | AGTTTTTGATTCTGCGGTTCCAGTGGTTATCAGTTTCGATGATAATTACG |
| | | CAGTCAGCGGTGGTGCATTAATTAATTCCATTGTCCGGCATGCGGATAAA |
| | | AATAAAAATTATGATATCGTCGTACTCGAAAACAAAGTAAGCTATTTGAA |
| | | TAAAACGCGGTTAGTAAATCTAACCTCGGCTCATCCGAATATTTCTCTTC |
| | | GTTTTTTTGACGTTAATGCTTTCACTGAAATAAACGGTGTGCATACCCGA |
| | | GCGCATTTTAGCGCATCAACGTATGCCCGTCTTTTTATTCCTCAACTGTT |
| | | CAGACGATACGATAAAGTCGTATTTATTGATTCGGATACCGTTGTAAAGG |
| | | CTGACCTGGGTGAACTGCTTGATGTCCCTCTGGGCAACAATTTAGTTGCA |
| | | GCGGTTAAGGATATCGTCATGGAAGGTTTTGTAAAATTTTCTGCAATGTC |
| | | GGCATCAGATGATGGCGTTATGCCGGCAGGCGAATATTTACAGAAAACCT |
| | | TAAACATGAATAACCCTGATGAATATTTTCAGGCAGGGATTATTGTTTTT |
| | | AATGTCAAACAAATGGTCGAAGAAAATACTTTTGCTGAATTGATGCGGGT |
| | | ATTAAAGGCAAAAAAATACTGGTTCCTCGACCAGGATATCATGAATAAAG |
| | | TTTTCTACTCTCGAGTCACATTTCTGCCATTAGAGTGGAACGTTTATCAT |
| | | GGTAATGGCAACACGGATGATTTCTTCCCTAATCTTAAGTTTGCAACGTA |
| | | TATGAAATTTTTAGCAGCTCGCAAGAAGCCTAAAATGATTCATTATGCGG |
| | | GTGAGAACAAACCATGGAATACCGAAAAAGTCGATTTTTATGACGACTTT |
| | | ATTGAAAACATCGCTAACACTCCATGGGAGATGGAAATCTATAAACGTCA |
| | | GATGTCGTTAGCGGCTTCGATTGGTTTAACCCATAGCGAGCCGCAACAAC |
| | | AAATCTTGTTCCAGACCAAAATCAAGAACGTACTGATGCCTTATGTTAAT |
| | | AAATATGCACCAATAGGCACGCCAAGAAGAAACATGATGACTAAATATTA |
| | | TTACAAAGTACGCCGTGCTATTCTTGGATAATAAAAGAGACAACAGATGA |
| | | AAAGTAAAAAAATATTGATCGTAGGTGCTGGCTTCTCTGGTGCAGTTATC |
| | | GGTCGCCAACTTGCTGAGAAGGGACATCAAGTCCATATTATCGATCAGCG |
| | | TGATCATATTGGGGGGAATTCCTATGATGCACGGGACGCTGAAACGAATG |
| | | TGATGGTACATGTTTATGGACCCCATATTTTCCATACTGACAATGAAACA |
| | | GTGTGGAACTATGTCAACAAGCATGCAGAGATGATGCCCTATGTGAACCG |
| | | GGTTAAAGCGACAGTTAATGGTCAGGTATTTTCCCTGCCTATTAATTTGC |
| | | ATACTATCAATCAGTTTTTCTCAAAAACTTGTTCGCCTGATGAGGCCAGA |
| | | GCGCTCATTGCTGAGAAAGGGGACAGCACTATTGCTGATCCACAAACTTT |
| | | TGAAGAGCAAGCGTTACGCTTTATTGGTAAAGAGTTATATGAGGCCTTTT |
| | | TTAAAGGATATACGATTAAACAGTGGGGGATGCAACCCTCGGAACTGCCC |
| | | GCATCTATTCTTAAACGTCTTCCTGTTCGTTTTAACTATGATGATAATTA |
| | | TTTTAACCACAAATTTCAGGGCATGCCGAAATGTGGTTATACGCAGATGA |
| | | TTAAGTCCATTCTCAATCATGAAAATATCAAGGTTGACTTACAGCGGGAA |
| | | TTTATCGTTGAAGAGCGAACTCATTACGATCACGTATTCTATAGCGGTCC |
| | | ATTAGATGCGTTTTATGGCTACCAATATGGCCGTCTGGGCTATCGAACAT |
| | | TAGATTTTAAAAAGTTTACCTATCAGGGTGATTACCAGGGCTGCGCAGTG |
| | | ATGAACTATTGTTCTGTGGATGTGCCCTATACTCGCATCACTGAACATAA |
| | | ATATTTTTCTCCCTGGGAACAACACGACGGCTCTGTTTGTTATAAAGAAT |
| | | ATAGCCGTGCTTGTGAAGAAAATGATATTCCTTACTATCCTATTCGCCAG |
| | | ATGGGAGAGATGGCTCTTCTTGAAAAATATTTGTCATTGGCCGAGAATGA |
| | | AACCAACATCACTTTTGTCGGTCGTCTTGGAACCTACCGTTACCTTGATA |
| | | TGGATGTGACCATCGCCGAAGCATTGAAAACGGCAGAAGTCTATTTAAAT |
| | | TCACTCACTGAAAATCAGCCAATGCCTGTGTTTACGGTTTCTGTACGATG |
| | | AAATATACGGCATTGATAGTGACATTCAATCGTCTCGGCAAACTGAAAAA |
| | | AACGGTTGAAGAGACCCTCAAACTTGAATTCACTAATATTGTTATTGTCA |
| | | ATAACGGGTCCACGGATGGGACCCAAGCCTGGCTTTCGTCAATTGTTGAT |
| | | ACACGAGTCATTGTATTAACCCTCACCGAGAATACCGGTGGGGCGGGGGG |
| | | CTTTAAAACCGGTAGTCAGTATATCTGTGAACAGCTGGCAAGTGATTGGG |
| | | TATTTTTCTACGATGACGATGCTTACCCCTATCCAGACACGTTGAAGTCC |
| | | TTTTCACAGCTGGATAAGCAGGGATGTCGGGTATTTAGTGGACTGGTGAA |
| | | AGATCCGCAAGGAAAACCGTGTCCGATGAATATGCCGTTCTCGCGTGTGC |
| | | CAACTTCACTTGGCGACACTGTACGCTATTTACGCTACCCTGGAGAGTTT |
| | | ATCCCGGCAGCTAATCGTTCTATGTTCGTACAAACGGTTTCATTTGTTGG |
| | | GATGGTCATACATCGTGATCTGCTCACGACCAGCCTTGACCACATCCATG |
| | | AACAGCTTTTTATCTACTTTGATGATCTTTACTTTGGCTATCAGCTATCA |
| | | CTAGCTGGTGAGAAAATTATGTATAGCCCAGAGTTGCTTTTTTATCATGA |
| | | TGTGAGTATTCAGGGCAAACTTATTGCACCTGAATGGAAGGTTTACTATC |
| | | TATGCCGTAATTTGATCCTGTCGAAGAAAATATTCCAGAAAAATGGCGTG |
| | | TATAGCAATTCAGCGATAGCGATACGCATCCTAAAATATATATTAATCCT |
| | | GCCATGGCAACGTCAAAAATATTCCTATATGAAATTTATTCTTCGTGGAA |
| | | TTTCACATGGCATAAAAGGTATTAGTGGTAAGTATCATTAAGTGGGCATA |
| | | GCAATGAGAAAATTGTGTTATTTCATAAATTCGGATTGGTACTTCGATTT |
| | | ACACTGGATCGATCGTGCCATCGCCTCCCGTGATGCAGGTTATGAGATTC |
| | | ACATCATCAGCCATTTTATTGATGACAACATAATAAATAAATTCAAAACA |
| | | TTCGGCTTTATTTGCCATAATGTTACTCTTGATGCTCAATCTTTTAATGC |
| | | ATTAGTTTTCTTTCGTACTTACCATGATGTGCAAAAAATTATTAAAAATA |
| | | TAAAACCGGATCTCTTGCATTGCATTACTATCAAGCCATGTTTGATTGGT |
| | | GGTGTGCTCGCGAAGAAATTTAATCTGCCGGTCATCGTAAGTTTTGTTGG |
| | | GCTTGGAAGAGTATTTTCTTCAGACAGCATGCCTTTAAAATTATTGCGGC |
| | | AGTTTACTATTGCTGCATATAAATATATTGCCAGTAATAAGCGCTGTATA |
| | | TTTATGTTTGAACATGACCGCGACAGAAAAAAACTGGCTAAGTTGGTTGG |
| | | ACTCGAAGAACAACAGACTATTGTTATTGATGGTGCAGGCATTAATCCAG |
| | | AGATATACAAATATTCTCTTGAACAGAATCACGATGTCCCTGTTGTATTG |
| | | TTTGCCAGCCGTATGTTGTGGAGTAAAGGACTGGGCGACTTAATTGAAGC |
| | | GAAGAAAATATTACGCAGTAAGAATATTCACTTTACTTTGAATGTTGCTG |
| | | GAATTCTGGTCGAAAATGATAAAGATGCAATTTCCCTTCAGGTCATTGAA |
| | | AATTGGCATCAGCAAGGATTAATTAACTGGTTAGGTCGTTCGAATAACGT |
| | | TTGCGATCTTATTGAGCAATCAAATATCGTTGCTTTGCCGTCAGTTTATT |
| | | CTGAAGGTGTTCCGCGAATTCTTCTGGAAGCATCTTCTGTGGGTCGCGCT |
| | | TGTATTGCTTATGATGTTGGTGGTTGTGATAGCCTTATTATTGATAACGA |
| | | TAATGGAATTATTGTTAAAAGCAATTCACCTGAAGAGCTGGCTGATAAAC |
| | | TTGCCTTTTTGCTTAGCAATCCTAAAGCACGTGTTGAAATGGGTATTAAA |
| | | GGACGTAAGCGTATTCAGGATAAATTCTCGAGCGGGATGATTATCAGTAA |
| | | GACGCTAAAGACTTATCATGATGTGGTTGAGGGATAGTTGTCGATCAAAC |
| | | GGTTATCCTTTTTTATTAATTGCCAGATATTGTTTCTTTACCATCAAATT |
| | | TTTTTTGAAGTATATTATTAACTAAAATTACTGTAACGTGTCACTTGGGA |
| | | GGCGATCAAATGTCTGAAAGATCTTCAAGTGCACTGGTCTCTGTTGTGAT |
| | | ACCTGTGCACGATGCTGCAGAATATATATCTGATACGCTAAGTTCCATTT |
| | | TATCGCAATCGTTACAGGATATTGAAGTCATCATTATTGATGACAATTCA |
| | | GCTGATGATACGTTAAAGCTACTGCAGTCCTTTGCCGCTAATGACTCGCG |
| | | AATACGTCTTTTGAATAATTCGCAGAATATCGGTGCAGGTGCATCACGTA |
| | | ACATGGGGTTAAAAATAGCAAGTGGCGAATATATCATTTTTCTTGATGAT |
| | | GACGATTATGCCGATGCTAATATGCTCAAACGGATGTATGATCATGCTGC |
| | | ATTGCTGCAAGCCGATGTGGTTATCTGCCGATGCCAGTCTTTAGATCTAC |
| | | AAACCCATTCATATGCACCAATGCCATGGTCTGTGCGCGTAGATTTACTC |
| | | CCCCAAAAAGAACTATTTTCATCAGATGAAATTACTCATAATTTCTTTGA |
| | | TGCATTTATCTGGTGGCCCTGGGATAAGCTTTTCCGTCGCCAGGCTATAC |
| | | TGGATACTGGGTTACAATTCCAGGATTTAAGAACGACTAATGATTTATTT |
| | | TTTGTTAGCGCTTTTATGCTACTTACCAAAAGAATGGCGTTCCTGGATGA |
| | | GATCTTGATTTCTCATTCCATTAACCGCAGTGGTTCATTATCGGTGACCA |
| | | GAGAGAAATCATGGCACTGTGCTCTTGATGCGTTACGTGCCCTCTATTCC |
| | | TTTATTGACTCAAAGCACTTGTTGCCTTCACGTGGTAGAGACTTTAATAA |
| | | TTATGCAGTGACTTTTCTTGAGTGGAATTTAAATACGATTTCTGGTCCGG |
| | | CGTTTGATTCTTTATTCACTGCTTCACGCGAATTCATCGCCTCATTGGAT |
| | | ATTGATGAAAGCGATTTTTATGATGATTTTATCAAAGCGGCACACTATCG |
| | | CCTGATTCGATTAACGCCGGAAGAGTATCTTTTCTCGTTAAAAGATCGGG |
| | | TATTACATGAGCTTGAATCCTCTAATCTATCTACAGAGAAGTTGCAAGCC |
| | | AGTATTGCTTCTCAGGATCAAGTTCTTAAAGCCAGGGAAGAAGAAATTGA |
| | | TGAGCTAAGAGCGTCCGTTGCACAGAAAAAAGAACGTATTGATAGGCTGA |
| | | TGGAGCGAAATGCATATTTAGAGACTGAGTATCAGAAACAGCAAGATCAA |
| | | TTAACTAAACTACAAAATGAATTAAATAACGCTGCTCAACGTTATTCAGC |
| | | CCTTATTTCATCATTGTCATGGAAAGTTACAAGACCTTTAAGGTTAATCA |
| | | AAGCGTTAATCGTGAAGAAAATGTAATATTTTTATCAATAATTCATGCTT |
| | | ATTTTAGATGCAGAGAGATACTCCTGATTAACGAGAAAAGTTTTGCAGGG |
| | | AGGTATATTAACACCTCCCTTTGTTATTATTACTTATGCCGTGCTCTTAA |
| | | ATTATCAATCACTTC |
| 14 | 11.1kb v2 | ATGAGTATAAAGATGAAGTACAATTTAGGGTATTTATTTGATTTACTTGT |
| | operon | TGTGATAACAAATAAAGATCTAAAAGTGCGCTATAAGAGCAGCATGCTAG |
| | (Gal III | GCTATTTATGGTCAGTAGCAAATCCATTGCTTTTTGCCATGATTTATTAT |
| | biosynthetic | TTTATATTTAAGCTGGTAATGAGAGTACAAATTCCAAATTATACAGTTTT |
| | gene cluster) | CCTCATTACCGGCTTGTTTCCGTGGCAATGGTTTGCCAGTTCGGCCACTA |
| | | ACTCATTATTTTCATTCATCGCTAACGCTCAAATTATCAAGAAGACAGTT |
| | | TTTCCCCGGTCCGTGATTCCGCTAAGTAATGTAATGATGGAAGGGTTGCA |
| | | TTTTCTTTGTACCATCCCGGTTATTGTTGTCTTTCTTTTTGTTTATGGCA |
| | | TGACGCCGTCCTTGTCCTGGGTTTGGGGTATACCTCTCATTGCTATTGGC |
| | | CAGGTGATTTTCACCTTTGGTGTTTCAATCATCTTTTCAACGCTGAACCT |
| | | GTTTTTCCGTGACCTGGAGCGCTTTGTCAGTCTGGGGATTATGCTGATGT |
| | | TTTATTGTACGCCGATTTTATATGCGTCTGATATGATTCCGGAAAAATTT |
| | | AGCTGGATAATTACCTACAATCCGCTAGCGAGTATGATTCTTAGTTGGCG |
| | | TGATTTATTCATGAATGGGACTCTTAATTATGAGTATATTTCTATACTCT |
| | | ATTTTACGGGAATTATTTTGACGGTTGTCGGTTTGTCTATTTTCAATAAA |
| | | TTAAAATATCGATTTGCAGAGATCTTGTAATGCACCCAGTTATTAACTTC |
| | | AGTCATGTTACAAAAGAGTATCCTCTGTACCATCATATTGGCTCAGGAAT |
| | | CAAAGATTTAATTTTCCATCCGAAACGCGCTTTTCAATTGCTGAAGGGGC |
| | | GGAAATATTTAGCTATCGAAGACGTATCCTTTACAGTTGGCAAAGGTGAG |
| | | GCTGTTGCTCTGATTGGACGTAATGGGGCAGGAAAGAGTACCTCTCTTGG |
| | | CCTGGTTGCCGGCGTGATTAAGCCAACTAAGGGAACCGTCACCACTGAAG |
| | | GACGGGTGGCATCGATGCTTGAACTCGGCGGAGGCTTTCATCCGGAACTT |
| | | ACCGGGCGTGAGAATATTTACCTGAATGCTACTCTGCTGGGCCTTCGGCG |
| | | TAAAGAGGTCCAGCAACGTATGGAACGTATTATTGAATTTTCGGAACTGG |
| | | GAGAATTCATAGACGAGCCAATCAGAGTGTACTCAAGCGGAATGCTAGCT |
| | | AAGTTAGGTTTTTCGGTCATCAGTCAAGTTGAACCGGATATTTTAATTAT |
| | | TGATGAAGTTCTTGCAGTAGGTGATATCGCTTTTCAGGCAAAATGTATTA |
| | | AGACCATCAGAGATTTTAAGAAAAGAGGCGTGACAATATTGTTTGTTAGC |
| | | CACAATATGAGTGACGTTGAAAAAATCTGCGACAGAGTCATCTGGATCGA |
| | | AAATCATAGGCTCAGAGAAGTGGGGTCTGCAGAGCGAATCATTGAACTGT |
| | | ACAAGCAAGCAATGGCTTAATCAGTGGGTAATATAATGAACAATAGCGTT |
| | | AAAATCTATACCAGCCACCATAAGCCTAGTGCTTTTCTTAATGCTGCAAT |
| | | TATCAAACCTCTGCATGTCGGCAAAGCTAATTCTTGTAATGAAATTGGTT |
| | | GTCCAGGAGATGACACTGGCGATAATATTTCCTTTAAGAATCCGTTTTAT |
| | | TGCGAACTAACTGCGCATTATTGGGTTTGGAAAAACGAAGAGCTGGCAGA |
| | | CTATGTCGGTTTCATGCACTATCGCCGTCATCTTAATTTTTCCGAAAAAC |
| | | AAACTTTTTCTGAGGATACCTGGGGGGTCGTGAACCATCCATGCATTGAT |
| | | GAAGAATATGAGAAGATCTTTGGATTAAACGAAGAAACAATTCAACGGTG |
| | | TGTCGAAGGTATTGACATCTTGCTGCCCAAAAAATGGTCTGTCACTGCGG |
| | | CGGGAAGTAAAAATAATTACGATCACTATGAACGAGGTGAATACTTACAC |
| | | ATTCGTGATTATCAGGCTGCCATTGCCATCGTTGAAAAACTATATCCAGA |
| | | GTATAGCACGGCAATAAAAACGTTTAATGATGCCAGTGATGGCTATTACA |
| | | CAAATATGTTTGTCATGCGCAAAGATATTTTTGTTGACTATTCTGAGTGG |
| | | CTCTTTTCCATTCTGGATAATCTCGAAGATGCCATCTCGATGAACAATTA |
| | | TAATGCTCAGGAAAAACGCGTTATTGGGCATATAGCAGAACGGCTGTTTA |
| | | ATATTTACATTATTAAGCTGCAACAAGATGGTGAGCTTAAGGTAAAAGAA |
| | | TTACAGCGTACTTTTGTCAGCAATGAAACATTCAATGGTGCACTGAATCC |
| | | AGTTTTTGATTCTGCGGTTCCAGTGGTTATCAGTTTCGATGATAATTACG |
| | | CAGTCAGCGGTGGTGCATTAATTAATTCTATTGTCCGGCATGCGGATAAA |
| | | AATAAAAATTATGATATCGTCGTACTCGAAAACAAAGTAAGCTATTTGAA |
| | | TAAAACGCGGTTAATAAATCTAACCTCGGCTCATCCGAATATTTCTCTTC |
| | | GTTTTTTTGACGTTAATGCCTTCACTGAAATAAACGGTGTGCATACCCGA |
| | | GCGCATTTTAGCGCATCAACGTATGCCCGTCTTTTTATTCCTCAACTGTT |
| | | CAGACGATACGATAAAGTCGTATTTATTGATTCGGATACCGTTGTAAAGG |
| | | CTGACCTGGGTGAACTGCTTGATGTCCCTCTGGGCAACAATTTAGTTGCA |
| | | GCGGTTAAGGATATCGTCATGGAAGGTTTTGTAAAATTTTCTGCAATGTC |
| | | GGCATCAGATGATGGCGTTATGCCGGCAGGCGAATATTTAAAAAAAACCT |
| | | TAAACATGAATAACCCTGATGAATATTTTCAGGCAGGGATTATTGTTTTT |
| | | AATGTCAAACAAATGGTCGAAGAAAATACTTTTGCTGAATTGATGCGGGT |
| | | ATTAAAGGCAAAAAAATACTGGTTCCTCGACCAGGATATCATGAATAAAG |
| | | TCTTCTACTCTCGAGTCACATTTCTGCCATTAGAGTGGAACGTTTATCAT |
| | | GGTAATGGCAACACGGATGATTTCTTCCCTAATCTTAAGTTTGCAACGTA |
| | | TATGAAATTTTTAGCAGCTCGCAAGAAGCCTAAAATGATTCATTATGCGG |
| | | GTGAGAACAAACCATGGAATACCGAAAAAGTCGATTTTTATGACGACTTT |
| | | ATTGAAAACATCGCTAACACTCCATGGGAGATGGAAATCTATAAACGTCA |
| | | AATGTCGTTAGCGGCTTCGATTGGTTTAACCCATAGCGAGCCGCAACAAC |
| | | AAATCTTGTTCCAGACCAAAATCAAGAACGTACTGATGCCTTATGTTAAT |
| | | AAATATGCACCAATAGGCACGCCAAGAAGAAACATGATGACTAAATATTA |
| | | TTACAAAGTACGCCGTGCTATTCTTGGATAATAAAAGAGACAACAGATGA |
| | | AAAGAAAAAAAATATTGATCGTAGGCGCTGGTTTCTCTGGTGCAGTTATC |
| | | GGTCGCCAACTTGCTGAGAAGGGACATCAAGTCCATATTATCGATCAGCG |
| | | TGATCATATTGGGGGGAATTCCTATGATGCACGCGACTCTGAAACGAATG |
| | | TGATGGTACATGTTTATGGACCCCATATTTTCCATACTGACAATGAAACA |
| | | GTGTGGAACTATGTCAACAAGCATGCAGAGATGATGCCCTATGTGAACCG |
| | | GGTTAAAGCGACAGTTAATGGTCAGGTATTTTCCCTGCCTATTAATTTGC |
| | | ATACTATCAATCAGTTTTTCTCAAAAACTTGTTCGCCTGATGAGGCCAGA |
| | | GCGCTCATTGCTGAGAAAGGGGACAGCACTATTGCTGATCCACAAACTTT |
| | | TGAAGAGCAAGCGTTACGCTTTATTGGTAAAGAGTTATATGAGGCCTTTT |
| | | TTAAAGGATATACGATTAAACAGTGGGGGATGCAACCCTCGGAACTGCCC |
| | | GCATCTATTCTTAAACGTCTTCCTGTTCGTTTTAACTATGATGATAATTA |
| | | TTTTAACCACAAATTTCAGGGCATGCCGAAATGTGGTTATACGCAGATGA |
| | | TTAAGTCAATTCTCAATCATGAGAATATCAAGGTTGACTTACAGCGGGAA |
| | | TTTATCGTTGACGAGCGAACTCATTACGATCACGTATTCTATAGCGGTCC |
| | | ATTAGATGCGTTTTATGGCTACCAATATGGCCGTCTGGGCTATCGAACAT |
| | | TAGATTTTAAAAAGTTTATCTATCAGGGTGATTACCAGGGATGCGCAGTG |
| | | ATGAACTACTGTTCTGTGGATGTGCCCTATACTCGCATCACTGAACATAA |
| | | ATATTTTTCTCCCTGGGAACAACACGACGGCTCTGTTTGTTATAAAGAGT |
| | | ATAGCCGTGCTTGTGAAGAAAATGATATTCCTTACTATCCTATTCGCCAG |
| | | ATGGGAGAGATGGCTCTTCTTGAAAAATATTTGTCATTGGCCGAGAATGA |
| | | AACCAACATCACTTTTGTCGGTCGTCTTGGAACCTACCGTTACCTTGATA |
| | | TGGATGTGACCATCGCCGAAGCATTGAAAACGGCAGAAGTCTATTTAAAT |
| | | TCACTCACTGAAAATCAGCCAATGCCTGTGTTTACGGTTTCTGTACGATG |
| | | AAATATACGGCATTGATAGTGACATTCAATCGTCTCGGCAAACTAAAAAA |
| | | AACGGTTGAAGAGACCCTCAAACTTGAATTCACTAATATTGTTATTGTCA |
| | | ATAACGGGTCCACGGATGGGACCCAAGCCTGGCTTTCGTCAATTGTTGAT |
| | | ACACGAGTCATTGTATTAACCCTCACCAAGAATACCGGTGGGGCGGGGGG |
| | | CTTTAAAACCGGTAGTCAGTATATCTGTGAACAGCTGGCAAGTGATTGGG |
| | | TATTTTTCTACGATGACGATGCTTACCCCTATCCAGACACGTTGAAGTCC |
| | | TTTTCACAGCTGGATAAGCAGGGATGTCGGGTATTTAGTGGACTGGTGAA |
| | | AGATCCGCAAGGAAAACCGTGTCCGATGAATATGCCGTTCTCGCGTGTGC |
| | | CAACTTCACTTGGCGACACTGTACGCTATTTACGCTACCCTGGAGAGTTT |
| | | ATCCCGGCAGCTAATCGTTCTATGTTCGTACAAACGGTTTCATTTGTTGG |
| | | GATGGTCATACATCGTGATCTGCTCGCGACCAGTCTTGACCACATCCATG |
| | | AACAGCTTTTTATCTACTTTGATGATCTTTACTTTGGCTATCAGCTATCA |
| | | CTAGCTGGTGAGAAAATTATGTATAGCCCGGAGTTGCTTTTTTATCATGA |
| | | TGTGAGTATTCAGGGCAAACTTATTGCACCTGAATGGAAGGTTTACTATC |
| | | TCTGCCGTAATTTGATCCTGTCGAAGAAAATATTCCAGAAAAATGCCGTG |
| | | TATAGCAATTCAGCGATAGCGATACGCATCCTAAAATATATATTAATCCT |
| | | GCCATGGCAACGTCAAAAATATTCCTATATGAAATTTATTCTTCGTGGAA |
| | | TTTCACATGGCATAAAAGGTATTAGTGGTAAGTATCATTAAGTGGGCATA |
| | | GCAATGAGAAAATTGTGTTATTTCATAAATTCGGATTGGTACTTCGATTT |
| | | ACACTGGATCGATCGTGCCATCGCCTCCCGTGATGCAGGTTATGAGATTC |
| | | ACATCATCAGCCATTTTATTGATGACAACATAATAAATAAATTCAAAACA |
| | | TTTGGCTTTATTTGCCATAATGTTACTCTTGATGCTCAATCTTTTAATGC |
| | | ATTAGTTTTCTTTCGTACTTACCATGATGTGCAAAAAATTATTAAAAATA |
| | | TAAAACCGGATCTCTTGCATTGCATCACTATCAAGCCATGTTTGATTGGT |
| | | GGTGTGCTCGCGAAGAAATTTAATCTGCCGGTCATCGTAAGTTTTGTTGG |
| | | GCTTGGAAGAGTATTTTCTTCTGACAGCATGCCTTTAAAATTATTGCGGC |
| | | AGTTTACTATTGCTGCATATAAATATATTGCCAGTAATAAGCGCTGTATA |
| | | TTTATGTTTGAACATGACCGCGACAGAAAAAAACTGGCTAAGTTGGTTGG |
| | | ACTCGAAGAACAACAGACTATTGTTATTGATGGTGCAGGCATTAATCCAG |
| | | AGATATACAAATATTCTCTTGAACAGGATCACGATGTCCCTGTTGTATTG |
| | | TTTGCCAGCCGTATGTTGTGGAGTAAAGGACTGGGCGACTTAATTGAAGC |
| | | GAAGAAAATATTACGCAGTAAGAATATTCACTTTACTTTGAATGTTGCTG |
| | | GAATTCTGGTCGAAAATGATAAAGATGCAATTTCCCTTCAGGTCATTGAA |
| | | AATTGGCATCAGCAAGGATTAATTAACTGGTTAGGTCGTTCGAATAATGT |
| | | TTGCGATCTTATTGAGCAATCAAATATCGTTGCTTTGCCGTCAGTTTATT |
| | | CTGAAGGTGTTCCGCGAATTCTTCTGGAAGCATCTTCTGTGGGTCGCGCT |
| | | TGTATTGCTTATGATGTTGGTGGTTGTGATAGCCTTATTATTGATAACGA |
| | | TAATGGAATTATTGTTAAAAGCAATTCACCTGAAGAGCTGGCTGATAAAC |
| | | TTGCCTTTTTACTTAGCAATCCTAAAGCACGCGTTGAAATGGGTATTAAG |
| | | GGGAGGAAACGTATACAAGATAAATTTTCTAGTGTTATGATTATCGATAA |
| | | AACATTGCAAATATATCATGATGTAGTTCGATGATGTGTAAGTTTCACAT |
| | | TTATTATTGCGAAAAACCTTCATATTGATAATAGTAATGTTTATATAATG |
| | | TAATTCAATTTACTACTAATGGTATTTTTATGGCTCATGAAAAAAGTGAT |
| | | ATAATTGTTTCGGTCGTTATTCCTGTTTACAACGCCGAAGAGTATATTGC |
| | | AGATACTCTAAAAAACATTGTTTCACAGTCATTGTATGAAATTGAAATTA |
| | | TAATAATCAATGATCATTCGAGTGATAATACATTAGATATCCTTAAGGAG |
| | | ATTGCATCCAGCGATGAAAGAATACGAATTATTGATAACGCTGTAAATAT |
| | | TGGAGCTGGCATATCACGTAATATAGGTCTTTCAGAAGCAAAGGGAGAAT |
| | | ATATAATATTTCTTGATGACGATGATTATGTCGATACGAACATGTTGAAG |
| | | CACATGTCTGATTGTGCGGAGCTATCAGGGGCAGATATCGTTGTATGCAG |
| | | AAGCCGCTCATTTAATCTACAATCTCTCCAGTATGCTCCAATGCCAGATT |
| | | CAATTCGAAAAGATTTATTACCTGAAAAAGCAGTTTTCTCGCCTGGAGAT |
| | | ATTGAGCGAGACTTTTTCAGGGCATTTATATGGTGGCCATGGGACAAACT |
| | | ATTCCGACGTGAATTTATTATTCAGCACTCGTTGAGCTACCAAGATTTAA |
| | | GAACATCAAATGATCTGTTTTTTGTGTGTGCATCTATGCTTAGTGCCGAA |
| | | AAGGTAACTATTCTTGATGAAATATTGATTACTCATACGATTAATCGAAA |
| | | AACATCATTGTCTTCAACTCGCTCCGTTTCCTATCATTGCGCACTTGATG |
| | | CTCTTGTTGCTCTAAGGGATTTTCTTTTTAAAAATGGCATGATGCAAAAG |
| | | CGACAAAGGGATTTTTATAATTACATTGTCGTATTCCTTGAGTGGCACTT |
| | | AAATACGCTATCGGGTGAAGCCTTTAATAAACTGTTTCAAGATGTCAAAT |
| | | TATTCATCAGCAGTTTTGATATCAATAATGAAGACTTTTATGATGAGTTT |
| | | ATTCTTTCTGCTTATCGACGAATCGCTGATATGTCTGCTGAAGAGTATCT |
| | | TTTTTCATTAAAAGATCGGGTTATTAATGAATTAGAGAATGCCCAACGAA |
| | | ATATTTTGACCTTACAAAACGAAGTTGAGGAGATAAAACAGCAGCTTCAA |
| | | CAAAAGGACGAAATGATTGCTTCTATGAATAGGGAAAATTTAGCTATTAA |
| | | AGCAGATAATAAAATTCTCGAAAATTACAATGAAGAACTAAAGACTGTTC |
| | | AGACAAAGTTTCTTAAACTACTCTCAAGTAAAGACTAGTATTTAAAAGCG |
| | | TATTTTATGATTACTGTAATAGCGCCCCCATAAAAAATGAGGGCGGCATA |
| | | GAAATTACTAATAATTTATCGTTGACCTTCGCATTGCATCTGACGTTTTA |
| | | ATAACCATACAATCATCAATACATCGATTGGTCTCAGAAATTATAACGTT |
| | | GTTAGATAGTTTGAAATTAAATCCTTTCATAATTCTGATCTCTTCAGCCA |
| | | TACCTTTGAGCGCCCAGGGCGCTGCTAATGAAATATTCCCAAACTCATCA |
| | | TATCTCTGTGTTTTTGTAAAGGCATTGTAAGCAGGATCTGTGAGATCGAA |
| | | CATTAATTTTCCTGTGTAATTCTTAGGTATTTTATTAGTTATTTCCGCAG |
| | | CAAGTGCCTGAATTTCAGAGCGTTGAGGAATAATAAATCCATTTATAATA |
| | | TTATACTGAGCTATTATCATAATTGTTAAAGCGATAAGAGGCCAGACAAA |
| | | TGCTTGCTTAGAAATTCTACTGACAAGGCTATTTATGCCAATAAGAAATA |
| | | GAGTTGATATAATAAGTTCTAAGGCCACTAACGAGCGGAATGCTGCCCAA |
| | | TTTTCTTTTGTCGCTAAATTTGGAGCGTAGGAACCTATCCCGATCGTTAT |
| | | GACTATGAACGTTTTCCATCTGCCTGTTTTTCCCACAAAAATAGTGTATA |
| | | AGCCGATTAAAATTGCAAATGAGGAGAACCAAGAATATATTTTTACTGGT |
| | | TGTATGTTATAGTTATTTACAGCGTTTATTAGTGATTCATTTATGAACCA |
| | | TTTCATCTTTCCACCGATATCTGCGGTTAACTCGGCTCTCGATAATGATT |
| | | CCCCATATAGCCAGACAGGAAGTACTTTTGACATGATAAAACTGCCTGCA |
| | | ACACCGATAACTAAAATGATAAAACATGTCGCAACTTTTTTCACAGTTAA |
| | | ACTACTTTCTTTTTTTATGCAACTATCAAGCATAAAAAAGAATAAGAATG |
| | | TAATTGCTGTCGGTTGATATATTGCAAATGCCACCCATAAGACAACAATG |
| | | GATGCTAATTTTTCTGGCAATGACGACCGCTGCTTCGAATGTGGGAAACA |
| | | TTTATTATAACTAATACCTGCCAGCAATACTGAAATAGTGAACGGGAAAC |
| | | ATGTTGCCCATGAAGCATAAACTTGAAACGCAGGGAGTAAGCAAATTAAC |
| | | AGCGGAAATATTATTTTGAATACGGGGTTATCAAATATTTTTCTGCTGTC |
| | | TATGAAGTTGTAAATAAAACAACTTAAGACAACAAGACTTAATATATTAA |
| | | AAAGCCGCAAATACGAAAATGAAGAAATATCATTAATTAACATTTTTCCA |
| | | TAGTAACGGAACACAGCATAAACGGGACGACCAGATTGGACATCCCACTG |
| | | AAACGAAGAGCCGTTTCTTGTTATAGCATCAAAGAGTGTTGACCAGTCGT |
| | | CTGAAAATGCATATGAAAAGAAAATTACCGGTGAAAATGTTAACATAAGC |
| | | AAAAAGAAATAAAAAATGTAAACGTTTTTTTTATTTCCCTCTGCTAAAGG |
| | | ATTGATCAGATTTTGCATGTTATTTTCCATTGCTATCATTACCTACGCTT |
| | | TCGTCAATGAAATATTTAGGTCTATTTTTCGTCTCTATATAAATTTTTCC |
| | | GACATATTCTCCTATAATACCTAAAGAAAGCATTTGCACGCCGCCAAGAA |
| | | AGAATATAGCGATCATGACTGATGTCCATCCCTCAACTGTAGTACCTGTT |
| | | GTTTTTTGAATTAAAGCATAAATCGCAGCGATGGTAGATATGATGCAAGT |
| | | TATAAAACCTGTCATAGCTATAATTCGTAACGGTGTAACTGATAATGAGG |
| | | TAATTCCCTCGAGAGCCAGCGCAAGCATTTTTTTAATTGGATATTTTGAT |
| | | TCACCGGCAATTCTTTCTTCACGGCTATATTGCACCTCGATCGAGGGGTA |
| | | TCCCACAAGAGGCACTAATCCACGTAAATATATATTTTGCTCTTTATATT |
| | | GTTTAAGAGCCTCCAATGCTCGATTACTTAATAATCGATAATCTGCATGA |
| | | TTTGGAGTTTGATTTACTCCCAAGTGGGACATTATTGCGTAAAATGCATT |
| | | AGCTGTTGTACGTTTAAAAAACGTGTCACTGTCTCGATTACCTCTTACGC |
| | | CGTATACTATGTCATATCCCTGGCTGTAAGCGTCAATCATTTTTTCGATG |
| | | CAATTTACATCGTCTTGTAGATCCGCATCGATGCTAATGGTTACGTCTGT |
| | | ATCGACCGAGCGTAACCCTGCCATCAACGCAATTTGATGTCCTTTATTTC |
| | | TTGATAATTTTATTCCTCGCACATAGTGATAAGCGGTCGAGGCATCTTTA |
| | | ATTTGTGCCCAAGTATTGTCACGACTACCATCATCGACAAACAAAAGATA |
| | | ACTATTGTTATTAATTTTATTTCTGGCTATCAATGAATTTAGTACATTCG |
| | | AAAGCTTTTCGAGACAGAAAGGAAAAGCCTCTTGTTCATTATAGCAAGGT |
| | | ACCACAATAGCTAAAGAAGGAGTGCTTTTTATATCAGTTGAGGTTGTCAT |
| | | TTCATCGCCCAGAACTTGTTTAAAATAAAACCTGTGATAGTGTATGTGAA |
| | | CATCCCAAGGATTTGTGCTGAATATATTTTTTCTGGCATAAAAACGAAAA |
| | | ATATTTTTATGACAATGATATTTGCCACATAACAAATGAAGCAAACACAT |
| | | AAAAATTTTATTAGTCTATTGATACTGATTGGTTGCGTAAATGTAAATAT |
| | | TGTGTTTGCTATAAAGCTGAAAACAATACCTACAACATAACCCATCGCAT |
| | | TGGACAGATAAATGCCAAGACCCAAATGCATTAGCAGGAAAATTACAACT |
| | | GCCGTAATTAGTGTATTGACTATCCCAACTAACCCATATTTCATTAGTTG |
| | | CCATAATGGGCCTGAACTTGGCATTATATACTCCGCTAGCGTTCCAATTG |
| | | GATGTTAAAAGCGGCAGCATTCTAACAAACTACATCTATCATGTGAATCC |
| | | AATTCACATCTCAAATATTAGGTTGTAAAGGATATTGGGAGGTATTTCGA |
| | | GTGCTGCGTGAAGGGTTCATTTAGAAAGAGTAATTAATGGCGGCTTTATA |
| | | ACCGCCATGTCTTATATTACCTATGCCGTGCTCTTAAATTATCAATCACT |
| | | TC |
| 15 | 3.4kb wbbZY | TGATTTAGCACTGCACTGAATTTGGGCCAGGGGCAAATCTGGCCGGGAAC |
| | fragment | TCAAAAATGCATGCAACTAAAACAGGGTTATTTACAGACAAATTTAAAAT |
| | (Gal II | TAGCTGAAAGTTAATATTATTTTTGCGGAGCCCTTTCGGGCCCCGAATAT |
| | biosynthetic | TACTTTATTTTAACATTGATTTCACTTTCCGGGCAACCCGGCGAACCAGG |
| | gene cluster) | CTGGTGCCTCGTTTTGCGCCTTGGACATGAATTGCTTCATACAGAGCATT |
| | | AAAACGGTCATGGGCCCAGCCATCTCTTTGCTGAGAAATAAGACCATCAA |
| | | CCTTATTATTCAAAAGTTGTAACTCGTTACTGACAGAATGACCACCATTG |
| | | ATGCTCCGCAAGCGATTCACTTCACTCTCAAGTTCTTGAATCTTCTCGAG |
| | | GTAGGAAGGTATCAACCCCATTGCATAGTTATATTTAACATTCTCATCCA |
| | | TAAAAGGACGGAAACTGCGGTAGTTATTTTTACTCTTATTTCCACTTAAC |
| | | AACATGCCGGAGTTTGCAACTCTATACCAAAATAGAGGTTCCGGGACGAT |
| | | TTGCAATTTATATCCCTGTAATGATATTTTGGCAAAAAACTCCCAGTCTT |
| | | CATGACCTAAACCGTAATCTTCAGTAAATCCGCCTACTTTTTCGAAAACC |
| | | TCTTTTCTGATCAGCGCATTAGCATCGCCAAAGCAGTTACTAAAGCTGGC |
| | | GATATTTAAATCAGGCCCTAACGGAAGCCAGCAGTGCGTCATTTTACGGA |
| | | ACGGAGAAGGGAACTCCTCACCAAAAATAAGATCGCTTGGTGTGGTTAAC |
| | | ACATCGGCCCCAGAGTTTAATGCTGCAGTAACAAACGTTTCTACCTCAAA |
| | | AGGCTTAGCAACATTATCATCGTCCATAAACATCAGATATTCGCCAGAGG |
| | | CGTGTCGCGCAGCCAAATTCCTTGCAGCACCCAGATAGTTATTAGAACTA |
| | | CGGACAATTTTCCAGCCTCGAGAGTTAAAATCATTCTCGATGAGATTCAA |
| | | ATAACGATGAGAATCTTCTGTCGTACTTCCATCATCAACCAAGATGACCT |
| | | CAATATTTTGGTACGTCTGAGATTTTATTGATGCGAGTGCTTGCTGAAGC |
| | | AAATGGTGACGTTCGAAGTGAGTTATACACACGCTAACTAACGGGCTGTT |
| | | AGCTTCATCGATTTTCTTGAATGCGCGGTTGTTTTTTCGTTCAACTGCGA |
| | | CAAACCAAGCTTCTTTAATATTGTCTTGTGATTCAGCAAGCCCTGGTTTT |
| | | ATATTTATATTTTTTAAGCGATAGTGGATTTTCCCGTATAAATCGACAGG |
| | | TGTAGGAATAAATAGAACTTCCGCATGATGCTCCTGCGGAATAAGCTCTG |
| | | GAATTCCACCAACGTTTGAAGCGAGGAAATTAACGTTATTAATCAAGCAT |
| | | TCATAAACAGTATAGGGTGAGTTTTCTACAAGTGATGGAATGATGACTAA |
| | | TACATTTTTTCTTTTTATATATTCATTAGCGTTGGTACGATCATAGTCGC |
| | | TGATGACATTAACTGCGAGTCCCAAATTTTTAGTCTGATTCATAATATAA |
| | | GTAAATGAATCAGTTTTCCCCATAGTGACATTTTTTCCGAGGAAGGTTAC |
| | | TCCAGAAATGCTCTCTTTATCTTCATCAGATAGTTTTCTTAATGCACGCA |
| | | GGAATATGTCAAGTCCTTTACGGGTTTCAAGGCGGCCGAAAAATACAAGC |
| | | TCAACGCCAGAAGCTGGCTTTTCATTTATTTTAACTGTAACATCATCTCT |
| | | CGTCACAAACCCTTGAAATGGCTCGCAATTTAAAATTACATGACGTTCTT |
| | | CAGGAACATTCCAGTGCTTACTCAACATCCAATCAATTAAATACTGAGAC |
| | | GGACTAACAACTTCATCCGCCATTTCAACCACCATTTTCTCCATATAATA |
| | | GAGTTCAAGATGGTTCTGATCATATGGAAGCTGGTAATTACCTTCATCAG |
| | | CCCATAACGTTGAACTGTGAGTATTTACAATGAACTTTGTATTTTCAAAA |
| | | TCCGTTCCATTCTTTTTGCTTAATAAAGTGTAATAAAGATCTGCCTGCCA |
| | | CTCACAAGAAATAACAGTGTCATAGATGTTATTTTCTTTCAACCAGAGAT |
| | | AAATTGAATAACTTTTCCTTCTAAAATACGGTGCATCAATATTAATCTCT |
| | | TTTATCAGTCCGGTTCTTAGCAGATTGATACCAAAGGTACTATAAATACG |
| | | TGACCAGTCGCTAAATTTCGATACAGATGATTCAGAATAGTCGCCACATG |
| | | TATACAATACATCAACATCATACCCCTTTTTTGCCAAAGTAGTGGCAAGG |
| | | GCAGTGAAAGCAGTTCCAATACCGCCGTTACGGACAGGCCCCTCAATGTC |
| | | CGGCGTCATTATAAGAATTTTCTTCATTGTAACCCTTCCTTTGTAACCTA |
| | | GACTTTTCTATGATATTAGTGAATTGAAGTAGTGTAAGATAGCAGTCGGT |
| | | AGCTTCTGTTAAACAGGATAAAAAATGACCAATATGAAGTTAAAATTTGA |
| | | TTTGCTTCTAAAATCTTATCATCTATCTCATCGATTTGTCTATAAGGCAA |
| | | ACCCTGGTAATGCTGGTGATGGTGTAATTGCATCTGCGACATATGACTTT |
| | | TTTGAACGAAATGCTCTTACCTATATCCCTTACAGAGATGGCGAGCGCTA |
| | | CAGTTCTGAAACTGATATTTTAATTTTTGGAGGCGGAGGAAACCTGATAG |
| | | AAGGATTGTATTCTGAAGGTCATGACTTTATCCAGAATAATATTGGGAAG |
| | | TTTCATAAAGTAATAATAATGCCGTCGACAATCAGAGGGTATAGCGATTT |
| | | ATTCATCAACAATATTGATAAGTTTGTTGTTTTTTGTCGCGAAAATATCA |
| | | CCTTCGATTATATTAAATCTCTCAACTACGAACCAAACAAGAACGTATTC |
| | | ATTACTGATGATATGGCATTTTATCTCGATCTTAATAAATACCTGTCACT |
| | | TAAACCCATCTATAAAAAACAGGCCAACTGCTTCAGAACGGACTCCGAAT |
| | | CTCTAACTGGAGACTATAAAGAAAACAATCATGATATTTCGCTCACCTGG |
| | | AATGGCGATTATTGGGATAATGAATTTCTGGCGCGTAATTCTACCCGTTG |
| | | CATGATAAACTTTCTTGAAGAGTATAAAGTTGTCAATACCGACAGGCTGC |
| | | ATGTGGCAATTTTAGCATCTCTGCTTGGCAAAGAAGTCAACTTCTATCCT |
| | | AACTCATATTACAAAAATGAAGCTGTTTACAATTATTCACTTTTTAATCG |
| | | TTATCCAAAAACATGCTTTATTACGGCAAGTTGAAAAAGGCAGCGTATAA |
| | | TAATACGCTGCCTGAAAGCCATATAACTGTTACAGCATTGTTAATTATTG |
| | | CCTGCCAGCCTTTAGGTGACTATTCATTCGCACGCCTATA |

1. A recombinant Escherichia coli (E. coli) host cell for producing a Klebsiella pneumoniae (K. pneumoniae) O-antigen, wherein the E. coli host cell comprises a polynucleotide encoding the K. pneumoniae O-antigen.

2. The recombinant E. coli host cell according to claim 1, wherein the K. pneumoniae O-antigen is selected from serotype O1 or serotype O2.

3. The recombinant E. coli host cell according to claim 2, wherein the K. pneumoniae O-antigen is selected from subtype v1 or subtype v2.

4. The recombinant E. coli host cell according to claim 3, wherein the K. pneumoniae O-antigen is selected from the group consisting of:
a) serotype O1 subtype v1 (O1v1),
b) serotype O1 subtype v2 (O1v2),
c) serotype O2 subtype v1 (O2v1), and
d) serotype O2 subtype v2 (O2v2).

5. The recombinant E. coli host cell according to claim 1, wherein the recombinant E. coli host cell is an E. coli O-antigen mutant strain.

6. The recombinant E. coli host cell according to claim 5, wherein the E. coli host cell is an E. coli K12 strain.

7. The recombinant E. coli host cell according to claim 4, wherein the polynucleotide encoding the K. pneumoniae O2v1 O-antigen comprises a gene cluster, wherein the gene cluster encodes:
a. Transport permease protein,
b. ABC transporter, ATP-binding component,
c. Glycosyltransferase,
d. UDP-galactopyranose mutase,
e. Galactosyltransferase (encoded by both wbbN and wbbO), and
f. FGlycosyltransferase family 2.

8. The recombinant E. coli host cell according to claim 4, wherein the polynucleotide encoding the K. pneumoniae O2v2 O-antigen comprises a gene cluster, wherein the gene cluster encodes:
a. Transport permease protein,
b. ABC transporter, ATP-binding component,
c. Glycosyltransferase,
d. UDP-galactopyranose mutase,
e. Galactosyltransferase (encoded by both wbbN and wbbO),
f. FGlycosyltransferase family 2,
g. protein encoded by gmIC (galactosyltransferase),
h. GmIB protein, and
i. GmIA protein.

9. The recombinant E. coli host cell according to claim 4, wherein the polynucleotide encoding the K. pneumoniae O1v1 O-antigen comprises:
a. a first gene cluster, wherein the first gene cluster encodes
i. Transport permease protein,
ii. ABC transporter, ATP-binding component,
iii. Glycosyltransferase,
iv. UDP-galactopyranose mutase,
v. Galactosyltransferase (encoded by both wbbN and wbbO), and
vi. FGlycosyltransferase family 2;
and
b. a second gene cluster, wherein the second gene cluster encodes
i. glycosyltransferase, and
ii. exopolysaccharide biosynthesis protein.

10. The recombinant E. coli host cell according to claim 4, wherein the polynucleotide encoding the K. pneumoniae O1v2 O-antigen comprises:
a. a first gene cluster, wherein the first gene cluster encodes
i. a. Transport permease protein,
ii. ABC transporter, ATP-binding component,
iii. Glycosyltransferase,
iv. UDP-galactopyranose mutase,
v. Galactosyltransferase (encoded by both wbbN and wbbO?),
vi. FGlycosyltransferase family 2,
vii. protein encoded by gmIC (please provide name),
viii. GmIB protein, and
ix. GmIA protein;
and
b. a second gene cluster, wherein the second gene cluster encodes
i. glycosyltransferase, and
ii. exopolysaccharide biosynthesis protein.

11. The recombinant E. coli host cell according to claim 4, wherein the polynucleotide encoding the K. pneumoniae O2v1 O-antigen comprises a gene cluster, wherein the gene cluster comprises the K. pneumoniae genes:
a. wzm,
b. wzt,
c. wbbM,
d. glf,
e. wbbN,
f. wbbO, and
g. kfoC.

12. The recombinant E. coli host cell according to claim 4, wherein the polynucleotide encoding the K. pneumoniae O2v2 O-antigen comprises a gene cluster, wherein the gene cluster comprises the K. pneumoniae genes:
a. wzm,
b. wzt,
c. wbbM,
d. glf,
e. wbbN,
f. wbbO,
g. kfoC,
h. gmIC,
i. gmIB, and
j. gmIA.

13. The recombinant E. coli host cell according to claim 4, wherein the polynucleotide encoding the K. pneumoniae O1v1 O-antigen comprises:
a. a first gene cluster, wherein the first gene cluster comprises the K. pneumoniae genes:
i. wzm,
ii. wzt,
iii. wbbM,
iv. glf,
v. wbbN,
vi. wbbO,
vii. kfoC;
and
b. a second gene cluster, wherein the second gene cluster comprises the K. pneumoniae genes:
i. wbbY, and
ii. wbbZ.

14. The recombinant E. coli host cell according to claim 4, wherein the polynucleotide encoding the K. pneumoniae O1v2 O-antigen comprises:
a. a first gene cluster, wherein the first gene cluster comprises the K. pneumoniae genes:
i. wzm,
ii. wzt,
iii. wbbM,
iv. gif,
v. wbbN,
vi. wbbO,
vii. kfoC,
viii. gmIC,
ix. gmIB, and
x. gmIA;
and
b. a second gene cluster, wherein the second gene cluster comprises the K. pneumoniae genes:
i. wbbY, and
ii. wbbZ.

15. The recombinant E. coli host cell according to claim 4, wherein the polynucleotide encoding the K. pneumoniae O2v1 O-antigen comprises a gene cluster, wherein the gene cluster comprises nucleotides having the nucleotide sequence set forth in SEQ ID NO: 13.

16. The recombinant E. coli host cell according to claim 4, wherein the polynucleotide encoding the K. pneumoniae O2v2 O-antigen comprises a gene cluster, wherein the gene cluster comprises nucleotides having the nucleotide sequence set forth in SEQ ID NO: 14.

17. The recombinant E. coli host cell according to claim 4, wherein the polynucleotide encoding the K. pneumoniae O1v1 O-antigen comprises:
a. a first gene cluster, wherein the first gene cluster comprises nucleotides having the nucleotide sequence set forth in SEQ ID NO: 13; and
b. a second gene cluster, wherein the second gene cluster comprises nucleotides having the nucleotide sequence set forth in SEQ ID NO: 15.

18. The recombinant E. coli host cell according to claim 4, wherein the nucleotide encoding the K. pneumoniae O1v2 O-antigen comprises:
a. a first gene cluster, wherein the first gene cluster comprises nucleotides having the nucleotide sequence set forth in SEQ ID NO: 14; and
b. a second gene cluster, wherein the second gene cluster comprises nucleotides having the nucleotide sequence set forth in SEQ ID NO: 15.

19. The recombinant E. coli host cell according to claim 4, wherein the polynucleotide encoding the K. pneumoniae O2v1 O-antigen comprises a gene cluster, wherein the gene cluster comprises nucleotides encoding the polypeptides having the amino acid sequences set forth in SEQ ID NOS: 1-7 or a fragment thereof.

20. The recombinant E. coli host cell according to claim 4, wherein the polynucleotide encoding the K. pneumoniae O2v2 O-antigen comprises a gene cluster, wherein the gene cluster comprises nucleotides encoding the polypeptides having the amino acid sequences set forth in SEQ ID NOs: 1-10 or a fragment thereof.

21. The recombinant E. coli host cell according to claim 4, wherein the polynucleotide encoding the K. pneumoniae O1v1 O-antigen comprises:
a. a first gene cluster, wherein the first gene cluster comprises nucleotides encoding the polypeptides having the amino acid sequences set forth in SEQ ID NOs: 1-7 or a fragment thereof; and
b. a second gene cluster, wherein the second gene cluster comprises nucleotides encoding the polypeptides having the amino acid sequences set forth in SEQ ID NOs: 11-12 or a fragment thereof.

22. The recombinant E. coli host cell according to claim 4, wherein the polynucleotide encoding the K. pneumoniae O1v2 O-antigen comprises:
a. a first gene cluster, wherein the first gene cluster comprises nucleotides encoding the polypeptides having the amino acid sequences set forth in SEQ ID NOs: 1-10; and
b. a second gene cluster, wherein the second gene cluster comprises nucleotides encoding the polypeptides having the amino acid sequences set forth in SEQ ID NOs: 11-12.

23. The recombinant E. coli host cell according to claim 1, wherein the polynucleotide sequence further encodes one or more primers.

24. The recombinant E. coli host cell according to claim 23, wherein the primer comprises at least 25 nucleic acid residues and at most 100 nucleic acid residues.

25. The recombinant E. coli host cell according to claim 24, wherein the primer comprises nucleic acids having the sequence selected from the group consisting of:
a. SEQ ID NO: 16 (wzm5′S2);
b. SEQ ID NO: 17 (hisl3′AS2);
c. SEQ ID NO: 18 (wzm5′S3);
d. SEQ ID NO: 19 (hisl3′AS3);
e. SEQ ID NO: 20 (pBAD33_O1O2S);
f. SEQ ID NO: 21 (pBAD33_O1O2AS);
g. SEQ ID NO: 22 (BAD18_O1O2S);
h. SEQ ID NO: 23 (pBAD18_O1O2AS);
i. SEQ ID NO: 24 (wbbZY PCR S1); and
j. SEQ ID NO: 25 (wbbZY PCR AS1).

26. The recombinant E. coli host cell according to claim 1, wherein the polynucleotide is integrated into a vector.

27. The recombinant E. coli host cell according to claim 26, wherein the vector is a plasmid.

28. The recombinant E. coli host cell according to claim 27, wherein the plasmid is selected from the group consisting of:
a. pBAD33;
b. pBAD18; and
c. Topo-blunt II.

29. The recombinant E. coli host cell according to claim 1, wherein the polynucleotide is integrated into the genomic DNA of the E. coli cell.

30. The recombinant E. coli host cell according to claim 29, wherein the polynucleotide is codon optimized for expression in the E. coli cell.

31. The recombinant E. coli host cell according to claim 1, wherein the polynucleotide comprises nucleotides encoding a gene cluster that is at least 75%, 80%, 85%, 90%, 95%, 96%, 97%, 98%, or 99% identical to SEQ ID NOs: 13-15 and 16-25 or a combination thereof.

32. A vector comprising a polynucleotide encoding a K. pneumoniae O-antigen.

33. The vector according to claim 32, wherein the K. pneumoniae O-antigen is selected from serotype O1 or serotype O2.

34. The vector according to claim 33, wherein the K. pneumoniae O-antigen is selected from subtype v1 or subtype v2.

35. The vector according to claim 34, wherein the K. pneumoniae O-antigen is selected from the group consisting of:
a) serotype O1 subtype v1 (O1v1),
b) serotype O1 subtype v2 (O1v2),
c) serotype O2 subtype v1 (O2v1), and
d) serotype O2 subtype v2 (O2v2).

36. The vector of claim 35, wherein the vector is a plasmid.

37. The recombinant E. coli host cell according to claim 36, wherein the plasmid is selected from the group consisting of:
a. pBAD33;
b. pBAD18; and
c. Topo-blunt II.

38. A culture comprising the recombinant E. coli host cell of claim 1, wherein said culture is at least 5 liters in size.

39. A method for producing a K. pneumoniae O-antigen, comprising
a. culturing a recombinant E. coli host cell according to claim 1 under a suitable condition, thereby expressing the K. pneumoniae O-antigen; and
b. harvesting the K. pneumoniae O-antigen produced by step (a).

40. The method according to claim 39, further comprising a step for purifying the K. pneumoniae O-antigen.


SEQUENCE LISTING (25 sequences)

SEQ ID NO: 1 - protein, 259 aa, Klebsiella pneumoniae
MSIKMKYNLGYLFDLLVVITNKDLKVRYKSSMLGYLWSVANPLLFAMIYYFIFKLVMRVQIPNYTVFLITGLFPWQWFASSATNSLFSFIANAQIIKKTVFPRSVIPLSNVMMEGLHFLCTIPVIVVFLFVYGMTPSLSWVWGIPLIAIGQVIFTFGVSIIFSTLNLFFRDLERFVSLGIMLMFYCTPILYASDMIPEKFSWIITYNPLASMILSWRDLFMNGTLNYEYISILYFTGIILTVVGLSIFNKLKYRFAEIL

SEQ ID NO: 2 - protein, 246 aa, Klebsiella pneumoniae
MHPVINFSHVTKEYPLYHHIGSGIKDLIFHPKRAFQLLKGRKYLAIEDVSFTVGKGEAVALIGRNGAGKSTSLGLVAGVIKPTKGTVTTEGRVASMLELGGGFHPELTGRENIYLNATLLGLRRKEVQQRMERIIEFSELGEFIDEPIRVYSSGMLAKLGFSVISQVEPDILIIDEVLAVGDIAFQAKCIQTIRDFKKRGVTILFVSHNMSDVEKICDRVIWIENHRLREVGSAERIIELYKQAMA

SEQ ID NO: 3 - protein, 631 aa, Klebsiella pneumoniae
MNNSVKIYTSHHKPSAFLNAAIIKPLHVGKANSCNEIGCPGDDTGDNISFKNPFYCELTAHYWVWKNEELADYVGFMHYRRHLNFSEKQTFSEDTWGVVNHPCIDEEYEKIFGLNEETIQRCVEGIDILLPKKWSVTAAGSKNNYDHYERGEYLHIRDYQAAIAIVEKLYPEYSAAIKTFNDASDGYYTNMFVMRKDIFVDYSEWLFSILDNLEDAISMNNYNAQEKRVIGHIAERLFNIYIIKLQQDGELKVKELQRTFVSNETFNGALNPVFDSAVPVVISFDDNYAVSGGALINSIVRHADKNKNYDIVVLENKVSYLNKTRLVNLTSAHPNISLRFFDVNAFTEINGVHTRAHFSASTYARLFIPQLFRRYDKVVFIDSDTVVKADLGELLDVPLGNNLVAAVKDIVMEGFVKFSAMSASDDGVMPAGEYLQKTLNMNNPDEYFQAGIIVFNVKQMVEENTFAELMRVLKAKKYWFLDQDIMNKVFYSRVTFLPLEWNVYHGNGNTDDFFPNLKFATYMKFLAARKKPKMIHYAGENKPWNTEKVDFYDDFIENIANTPWEMEIYKRQMSLAASIGLTHSEPQQQILFQTKIKNVLMPYVNKYAPIGTPRRNMMTKYYYKVRRAILG

SEQ ID NO: 4 - protein, 384 aa, Klebsiella pneumoniae
MKSKKILIVGAGFSGAVIGRQLAEKGHQVHIIDQRDHIGGNSYDARDAETNVMVHVYGPHIFHTDNETVWNYVNKHAEMMPYVNRVKATVNGQVFSLPINLHTINQFFSKTCSPDEARALIAEKGDSTIADPQTFEEQALRFIGKELYEAFFKGYTIKQWGMQPSELPASILKRLPVRFNYDDNYFNHKFQGMPKCGYTQMIKSILNHENIKVDLQREFIVEERTHYDHVFYSGPLDAFYGYQYGRLGYRTLDFKKFTYQGDYQGCAVMNYCSVDVPYTRITEHKYFSPWEQHDGSVCYKEYSRACEENDIPYYPIRQMGEMALLEKYLSLAENETNITFVGRLGTYRYLDMDVTIAEALKTAEVYLNSLTENQPMPVFTVSVR

SEQ ID NO: 5 - protein, 297 aa, Klebsiella pneumoniae
MKYTALIVTFNRLGKLKKTVEETLKLEFTNIVIVNNGSTDGTQAWLSSIVDTRVIVLTLTENTGGAGGFKTGSQYICEQLASDWVFFYDDDAYPYPDTLKSFSQLDKQGCRVFSGLVKDPQGKPCPMNMPFSRVPTSLGDTVRYLRYPGEFIPAANRSMFVQTVSFVGMVIHRDLLTTSLDHIHEQLFIYFDDLYFGYQLSLAGEKIMYSPELLFYHDVSIQGKLIAPEWKVYYLCRNLILSKKIFQKNGVYSNSAIAIRILKYILILPWQRQKYSYMKFILRGISHGIKGISGKYH

SEQ ID NO: 6 - protein, 377 aa, Klebsiella pneumoniae
MRKLCYFINSDWYFDLHWIDRAIASRDAGYEIHIISHFIDDNIINKFKTFGFICHNVTLDAQSFNALVFFRTYHDVQKIIKNIKPDLLHCITIKPCLIGGVLAKKFNLPVIVSFVGLGRVFSSDSMPLKLLRQFTIAAYKYIASNKRCIFMFEHDRDRKKLAKLVGLEEQQTIVIDGAGINPEIYKYSLEQNHDVPVVLFASRMLWSKGLGDLIEAKKILRSKNIHFTLNVAGILVENDKDAISLQVIENWHQQGLINWLGRSNNVCDLIEQSNIVALPSVYSEGVPRILLEASSVGRACIAYDVGGCDSLIIDNDNGIIVKSNSPEELADKLAFLLSNPKARVEMGIKGRKRIQDKFSSGMIISKTLKTYHDVVEG

SEQ ID NO: 7 - protein, 438 aa, Klebsiella pneumoniae
MSERSSSALVSVVIPVHDAAEYISDTLSSILSQSLQDIEVIIIDDNSADDTLKLLQSFAANDSRIRLLNNSQNIGAGASRNMGLKIASGEYIIFLDDDDYADANMLKRMYDHAALLQADVVICRCQSLDLQTHSYAPMPWSVRVDLLPQKELFSSDEITHNFFDAFIWWPWDKLFRRQAILDTGLQFQDLRTTNDLFFVSAFMLLTKRMAFLDEILISHSINRSGSLSVTREKSWHCALDALRALYSFIDSKHLLPSRGRDFNNYAVTFLEWNLNTISGPAFDSLFTASREFIASLDIDESDFYDDFIKAAHYRLIRLTPEEYLFSLKDRVLHELESSNLSTEKLQASIASQDQVLKAREEEIDELRASVAQKKERIDRLMERNAYLETEYQKQQDQLTKLQNELNNAAQRYSALISSLSWKVTRPLRLIKALIVKKM

SEQ ID NO: 8 - protein, 490 aa, Klebsiella pneumoniae
MENNMQNLINPLAEGNKKNVYIFYFFLLMLTFSPVIFFSYAFSDDWSTLFDAITRNGSSFQWDVQSGRPVYAVFRYYGKMLINDISSFSYLRLFNILSLVVLSCFIYNFIDSRKIFDNPVFKIIFPLLICLLPAFQVYASWATCFPFTISVLLAGISYNKCFPHSKQRSSLPEKLASIVVLWVAFAIYQPTAITFLFFFMLDSCIKKESSLTVKKVATCFIILVIGVAGSFIMSKVLPVWLYGESLSRAELTADIGGKMKWFINESLINAVNNYNIQPVKIYSWFSSFAILIGLYTIFVGKTGRWKTFIVITIGIGSYAPNLATKENWAAFRSLVALELIISTLFLIGINSLVSRISKQAFVWPLIALTIMIIAQYNIINGFIIPQRSEIQALAAEITNKIPKNYTGKLMFDLTDPAYNAFTKTQRYDEFGNISLAAPWALKGMAEEIRIMKGFNFKLSNNVIISETNRCIDDCMVIKTSDAMRRSTINY

SEQ ID NO: 9 - protein, 326 aa, Klebsiella pneumoniae
MTTSTDIKSTPSLAIVVPCYNEQEAFPFCLEKLSNVLNSLIARNKINNNSYLLFVDDGSRDNTWAQIKDASTAYHYVRGIKLSRNKGHQIALMAGLRSVDTDVTISIDADLQDDVNCIEKMIDAYSQGYDIVYGVRGNRDSDTFFKRTTANAFYAIMSHLGVNQTPNHADYRLLSNRALEALKQYKEQNIYLRGLVPLVGYPSIEVQYSREERIAGESKYPIKKMLALALEGITSLSVTPLRIIAMTGFITCIISTIAAIYALIQKTTGTTVEGWTSVMIAIFFLGGVQMLSLGIIGEYVGKIYIETKNRPKYFIDESVGNDSNGK

SEQ ID NO: 10 - protein, 125 aa, Klebsiella pneumoniae
MPSSGPLWQLMKYGLVGIVNTLITAVVIFLLMHLGLGIYLSNAMGYVVGIVFSFIANTIFTFTQPISINRLIKFLCVCFICYVANIIVIKIFFVFMPEKIYSAQILGMFTYTITGFILNKFWAMK

SEQ ID NO: 11 - protein, 740 aa, Klebsiella pneumoniae
MKKILIMTPDIEGPVRNGGIGTAFTALATTLAKKGYDVDVLYTCGDYSESSVSKFSDWSRIYSTFGINLLRTGLIKEINIDAPYFRRKSYSIYLWLKENNIYDTVISCEWQADLYYTLLSKKNGTDFENTKFIVNTHSSTLWADEGNYQLPYDQNHLELYYMEKMVVEMADEVVSPSQYLIDWMLSKHWNVPEERHVILNCEPFQGFVTRDDVTVKINEKPASGVELVFFGRLETRKGLDIFLRALRKLSDEDKESISGVTFLGKNVTMGKTDSFTYIMNQTKNLGLAVNVISDYDRTNANEYIKRKNVLVIIPSLVENSPYTVYECLINNVNFLASNVGGIPELIPQEHHAEVLFIPTPVDLYGKIHYRLKNINIKPGLAESQDNIKEAWFVAVERKNNRAFKKIDEANSPLVSVCITHFERHHLLQQALASIKSQTYQNIEVILVDDGSTTEDSHRYLNLIENDFNSRGWKIVRSSNNYLGAARNLAARHASGEYLMFMDDDNVAKPFEVETFVTAALNSGADVLTTPSDLIFGEEFPSPFRKMTHCWLPLGPDLNIASFSNCFGDANALIRKEVFEKVGGFTEDYGLGHEDWEFFAKISLQGYKLQIVPEPLFWYRVANSGMLLSGNKSKNNYRSFRPFMDENVKYNYAMGLIPSYLEKIQELESEVNRLRSINGGHSVSNELQLLNNKVDGLISQQRDGWAHDRFNALYEAIHVQGAKRGTSLVRRVARKVKSMLK

SEQ ID NO: 12 - protein, 269 aa, Klebsiella pneumoniae
MTNMKLKFDLLLKSYHLSHRFVYKANPGNAGDGVIASATYDFFERNALTYIPYRDGERYSSETDILIFGGGGNLIEGLYSEGHDFIQNNIGKFHKVIIMPSTIRGYSDLFINNIDKFVVFCRENITFDYIKSLNYEPNKNVFITDDMAFYLDLNKYLSLKPIYKKQANCFRTDSESLTGDYKENNHDISLTWNGDYWDNEFLARNSTRCMINFLEEYKVVNTDRLHVAILASLLGKEVNFYPNSYYKNEAVYNYSLFNRYPKTCFITAS

SEQ ID NO: 13 - DNA, 8215 bp, Artificial Sequence
  Synthetic nucleotide sequence
atgagtataaagatgaagtacaatttagggtatttatttgatttacttgttgtgataacaaataaagatctaaaagtgcgctataagagcagcatgctaggctatttatggtcagtagcaaatccattgctttttgccatgatttattattttatatttaagctggtaatgagagtacaaattccaaattatacagttttcctcattaccggcttgtttccgtggcaatggtttgccagttcggccactaactcattattttcattcatcgctaacgctcaaattatcaagaagacagtttttccccgttccgtgattccgctaagtaatgtgatgatggaaggcttgcattttctttgcaccatcccggttattgttgtctttctttttgtttatggcatgacgccgtccttgtcctgggtttggggtatacctctcattgctattggccaggtgattttcacctttggtgtttcaatcatcttttcaacgctgaacctgtttttccgtgacctggagcgctttgtcagtctggggattatgctgatgttttattgtacgccgattttatatgcgtctgatatgattccggaaaaatttagctggataattacctacaatccgctagcgagtatgattcttagttggcgtgatttattcatgaatgggactcttaattatgagtatatttctatactctattttacgggaatcattttgacggttgtcggtttgtctattttcaataaattaaaatatcgatttgcagagatcttgtaatgcacccagttattaacttcagtcatgttacaaaagagtatcctctgtaccatcatattggctcaggaatcaaagatttaattttccatccaaaacgcgcttttcagttgctgaaggggcggaaatatttagctatcgaagacgtatcctttacagttggcaaaggtgaggctgttgccctgattggacgtaatggggcaggaaagagtacctcgcttggcctggttgccggcgtgattaagccaactaagggaaccgtcaccactgaaggacgggtggcatcgatgcttgaactcggcggaggctttcatcctgaacttaccgggcgtgagaatatttacctgaatgctactctgctgggccttcggcgtaaagaggtccagcaacgtatggaacgtattattgaattttcggaactgggagaattcatagacgagccaatcagagtgtactcaagcggaatgctagctaagttaggtttttcggtcatcagtcaggttgaaccggatattttaattattgatgaagttctggcagtaggtgatatcgcttttcaggcaaaatgtattcagaccatcagagattttaagaaaagaggcgtgacaatattgtttgttagccacaatatgagtgacgttgaaaaaatctgcgacagagtcatctggatcgaaaatcataggctcagagaagtggggtctgcagagcgaatcattgaactgtacaagcaagcaatggcttaatcagtgggtaatataatgaacaatagcgttaaaatctataccagccaccataagcctagtgcttttcttaatgctgcaattatcaaacctctgcatgtcggcaaagctaattcttgtaatgaaattggttgtccaggagatgacactggcgataatatttcctttaagaatccgttttattgcgaactaactgcgcattattgggtttggaaaaacgaagagctggcagactatgtcggtttcatgcactatcgccgtcatcttaatttttccgaaaaacaaactttttctgaggatacctggggggtcgtgaaccatccatgcattgatgaagaatatgagaagatctttggattaaacgaagaaacaattcaacggtgtgtcgaaggtattgacatcttgctgcccaaaaaatggtctgtcactgcggcgggaagtaaaaataattacgatcactatgaacgaggtgaatacttacatattcgtgattatcaggctgccattgccatcgttgaaaaactatatccagagtatagcgcggcaataaaaacgtttaatgatgccagtgatggctattacacaaatatgtttgtcatgcgcaaagatatttttgttgactattctgagtggctcttttccattctggataatctcgaagatgctatctcgatgaacaattataatgctcaggaaaaacgcgttattgggcatatagcagaacggctgtttaatatttacattattaagttgcaacaagatggtgagcttaaggtaaaagaattacagcgtacttttgtcagcaatgaaacattcaatggtgcactgaatccagtttttgattctgcggttccagtggttatcagtttcgatgataattacgcagtcagcggtggtgcattaattaattccattgtccggcatgcggataaaaataaaaattatgatatcgtcgtactcgaaaacaaagtaagctatttgaataaaacgcggttagtaaatctaacctcggctcatccgaatatttctcttcgtttttttgacgttaatgctttcactgaaataaacggtgtgcatacccgagcgcattttagcgcatcaacgtatgcccgtctttttattcctcaactgttcagacgatacgataaagtcgtatttattgattcggataccgttgtaaaggctgacctgggtgaactgcttgatgtccctctgggcaacaatttagttgcagcggttaaggatatcgtcatggaaggttttgtaaaattttctgcaatgtcggcatcagatgatggcgttatgccggcaggcgaatatttacagaaaaccttaaacatgaataaccctgatgaatattttcaggcagggattattgtttttaatgtcaaacaaatggtcgaagaaaatacttttgctgaattgatgcgggtattaaaggcaaaaaaatactggttcctcgaccaggatatcatgaataaagttttctactctcgagtcacatttctgccattagagtggaacgtttatcatggtaatggcaacacggatgatttcttccctaatcttaagtttgcaacgtatatgaaatttttagcagctcgcaagaagcctaaaatgattcattatgcgggtgagaacaaaccatggaataccgaaaaagtcgatttttatgacgactttattgaaaacatcgctaacactccatgggagatggaaatctataaacgtcagatgtcgttagcggcttcgattggtttaacccatagcgagccgcaacaacaaatcttgttccagaccaaaatcaagaacgtactgatgccttatgttaataaatatgcaccaataggcacgccaagaagaaacatgatgactaaatattattacaaagtacgccgtgctattcttggataataaaagagacaacagatgaaaagtaaaaaaatattgatcgtaggtgctggcttctctggtgcagttatcggtcgccaacttgctgagaagggacatcaagtccatattatcgatcagcgtgatcatattggggggaattcctatgatgcacgggacgctgaaacgaatgtgatggtacatgtttatggaccccatattttccatactgacaatgaaacagtgtggaactatgtcaacaagcatgcagagatgatgccctatgtgaaccgggttaaagcgacagttaatggtcaggtattttccctgcctattaatttgcatactatcaatcagtttttctcaaaaacttgttcgcctgatgaggccagagcgctcattgctgagaaaggggacagcactattgctgatccacaaacttttgaagagcaagcgttacgctttattggtaaagagttatatgaggccttttttaaaggatatacgattaaacagtgggggatgcaaccctcggaactgcccgcatctattcttaaacgtcttcctgttcgttttaactatgatgataattattttaaccacaaatttcagggcatgccgaaatgtggttatacgcagatgattaagtccattctcaatcatgaaaatatcaaggttgacttacagcgggaatttatcgttgaagagcgaactcattacgatcacgtattctatagcggtccattagatgcgttttatggctaccaatatggccgtctgggctatcgaacattagattttaaaaagtttacctatcagggtgattaccagggctgcgcagtgatgaactattgttctgtggatgtgccctatactcgcatcactgaacataaatatttttctccctgggaacaacacgacggctctgtttgttataaagaatatagccgtgcttgtgaagaaaatgatattccttactatcctattcgccagatgggagagatggctcttcttgaaaaatatttgtcattggccgagaatgaaaccaacatcacttttgtcggtcgtcttggaacctaccgttaccttgatatggatgtgaccatcgccgaagcattgaaaacggcagaagtctatttaaattcactcactgaaaatcagccaatgcctgtgtttacggtttctgtacgatgaaatatacggcattgatagtgacattcaatcgtctcggcaaactgaaaaaaacggttgaagagaccctcaaacttgaattcactaatattgttattgtcaataacgggtccacggatgggacccaagcctggctttcgtcaattgttgatacacgagtcattgtattaaccctcaccgagaataccggtggggcggggggctttaaaaccggtagtcagtatatctgtgaacagctggcaagtgattgggtatttttctacgatgacgatgcttacccctatccagacacgttgaagtccttttcacagctggataagcagggatgtcgggtatttagtggactggtgaaagatccgcaaggaaaaccgtgtccgatgaatatgccgttctcgcgtgtgccaacttcacttggcgacactgtacgctatttacgctaccctggagagtttatcccggcagctaatcgttctatgttcgtacaaacggtttcatttgttgggatggtcatacatcgtgatctgctcacgaccagccttgaccacatccatgaacagctttttatctactttgatgatctttactttggctatcagctatcactagctggtgagaaaattatgtatagcccagagttgcttttttatcatgatgtgagtattcagggcaaacttattgcacctgaatggaaggtttactatctatgccgtaatttgatcctgtcgaagaaaatattccagaaaaatggcgtgtatagcaattcagcgatagcgatacgcatcctaaaatatatattaatcctgccatggcaacgtcaaaaatattcctatatgaaatttattcttcgtggaatttcacatggcataaaaggtattagtggtaagtatcattaagtgggcatagcaatgagaaaattgtgttatttcataaattcggattggtacttcgatttacactggatcgatcgtgccatcgcctcccgtgatgcaggttatgagattcacatcatcagccattttattgatgacaacataataaataaattcaaaacattcggctttatttgccataatgttactcttgatgctcaatcttttaatgcattagttttctttcgtacttaccatgatgtgcaaaaaattattaaaaatataaaaccggatctcttgcattgcattactatcaagccatgtttgattggtggtgtgctcgcgaagaaatttaatctgccggtcatcgtaagttttgttgggcttggaagagtattttcttcagacagcatgcctttaaaattattgcggcagtttactattgctgcatataaatatattgccagtaataagcgctgtatatttatgtttgaacatgaccgcgacagaaaaaaactggctaagttggttggactcgaagaacaacagactattgttattgatggtgcaggcattaatccagagatatacaaatattctcttgaacagaatcacgatgtccctgttgtattgtttgccagccgtatgttgtggagtaaaggactgggcgacttaattgaagcgaagaaaatattacgcagtaagaatattcactttactttgaatgttgctggaattctggtcgaaaatgataaagatgcaatttcccttcaggtcattgaaaattggcatcagcaaggattaattaactggttaggtcgttcgaataacgtttgcgatcttattgagcaatcaaatatcgttgctttgccgtcagtttattctgaaggtgttccgcgaattcttctggaagcatcttctgtgggtcgcgcttgtattgcttatgatgttggtggttgtgatagccttattattgataacgataatggaattattgttaaaagcaattcacctgaagagctggctgataaacttgcctttttgcttagcaatcctaaagcacgtgttgaaatgggtattaaaggacgtaagcgtattcaggataaattctcgagcgggatgattatcagtaagacgctaaagacttatcatgatgtggttgagggatagttgtcgatcaaacggttatccttttttattaattgccagatattgtttctttaccatcaaattttttttgaagtatattattaactaaaattactgtaacgtgtcacttgggaggcgatcaaatgtctgaaagatcttcaagtgcactggtctctgttgtgatacctgtgcacgatgctgcagaatatatatctgatacgctaagttccattttatcgcaatcgttacaggatattgaagtcatcattattgatgacaattcagctgatgatacgttaaagctactgcagtcctttgccgctaatgactcgcgaatacgtcttttgaataattcgcagaatatcggtgcaggtgcatcacgtaacatggggttaaaaatagcaagtggcgaatatatcatttttcttgatgatgacgattatgccgatgctaatatgctcaaacggatgtatgatcatgctgcattgctgcaagccgatgtggttatctgccgatgccagtctttagatctacaaacccattcatatgcaccaatgccatggtctgtgcgcgtagatttactcccccaaaaagaactattttcatcagatgaaattactcataatttctttgatgcatttatctggtggccctgggataagcttttccgtcgccaggctatactggatactgggttacaattccaggatttaagaacgactaatgatttattttttgttagcgcttttatgctacttaccaaaagaatggcgttcctggatgagatcttgatttctcattccattaaccgcagtggttcattatcggtgaccagagagaaatcatggcactgtgctcttgatgcgttacgtgccctctattcctttattgactcaaagcacttgttgccttcacgtggtagagactttaataattatgcagtgacttttcttgagtggaatttaaatacgatttctggtccggcgtttgattctttattcactgcttcacgcgaattcatcgcctcattggatattgatgaaagcgatttttatgatgattttatcaaagcggcacactatcgcctgattcgattaacgccggaagagtatcttttctcgttaaaagatcgggtattacatgagcttgaatcctctaatctatctacagagaagttgcaagccagtattgcttctcaggatcaagttcttaaagccagggaagaagaaattgatgagctaagagcgtccgttgcacagaaaaaagaacgtattgataggctgatggagcgaaatgcatatttagagactgagtatcagaaacagcaagatcaattaactaaactacaaaatgaattaaataacgctgctcaacgttattcagcccttatttcatcattgtcatggaaagttacaagacctttaaggttaatcaaagcgttaatcgtgaagaaaatgtaatatttttatcaataattcatgcttattttagatgcagagagatactcctgattaacgagaaaagttttgcagggaggtatattaacacctccctttgttattattacttatgccgtgctcttaaattatcaatcacttc

SEQ ID NO: 14 - DNA, 11052 bp, Artificial Sequence
  Synthetic nucleotide sequence
atgagtataaagatgaagtacaatttagggtatttatttgatttacttgttgtgataacaaataaagatctaaaagtgcgctataagagcagcatgctaggctatttatggtcagtagcaaatccattgctttttgccatgatttattattttatatttaagctggtaatgagagtacaaattccaaattatacagttttcctcattaccggcttgtttccgtggcaatggtttgccagttcggccactaactcattattttcattcatcgctaacgctcaaattatcaagaagacagtttttccccggtccgtgattccgctaagtaatgtaatgatggaagggttgcattttctttgtaccatcccggttattgttgtctttctttttgtttatggcatgacgccgtccttgtcctgggtttggggtatacctctcattgctattggccaggtgattttcacctttggtgtttcaatcatcttttcaacgctgaacctgtttttccgtgacctggagcgctttgtcagtctggggattatgctgatgttttattgtacgccgattttatatgcgtctgatatgattccggaaaaatttagctggataattacctacaatccgctagcgagtatgattcttagttggcgtgatttattcatgaatgggactcttaattatgagtatatttctatactctattttacgggaattattttgacggttgtcggtttgtctattttcaataaattaaaatatcgatttgcagagatcttgtaatgcacccagttattaacttcagtcatgttacaaaagagtatcctctgtaccatcatattggctcaggaatcaaagatttaattttccatccgaaacgcgcttttcaattgctgaaggggcggaaatatttagctatcgaagacgtatcctttacagttggcaaaggtgaggctgttgctctgattggacgtaatggggcaggaaagagtacctctcttggcctggttgccggcgtgattaagccaactaagggaaccgtcaccactgaaggacgggtggcatcgatgcttgaactcggcggaggctttcatccggaacttaccgggcgtgagaatatttacctgaatgctactctgctgggccttcggcgtaaagaggtccagcaacgtatggaacgtattattgaattttcggaactgggagaattcatagacgagccaatcagagtgtactcaagcggaatgctagctaagttaggtttttcggtcatcagtcaagttgaaccggatattttaattattgatgaagttcttgcagtaggtgatatcgcttttcaggcaaaatgtattaagaccatcagagattttaagaaaagaggcgtgacaatattgtttgttagccacaatatgagtgacgttgaaaaaatctgcgacagagtcatctggatcgaaaatcataggctcagagaagtggggtctgcagagcgaatcattgaactgtacaagcaagcaatggcttaatcagtgggtaatataatgaacaatagcgttaaaatctataccagccaccataagcctagtgcttttcttaatgctgcaattatcaaacctctgcatgtcggcaaagctaattcttgtaatgaaattggttgtccaggagatgacactggcgataatatttcctttaagaatccgttttattgcgaactaactgcgcattattgggtttggaaaaacgaagagctggcagactatgtcggtttcatgcactatcgccgtcatcttaatttttccgaaaaacaaactttttctgaggatacctggggggtcgtgaaccatccatgcattgatgaagaatatgagaagatctttggattaaacgaagaaacaattcaacggtgtgtcgaaggtattgacatcttgctgcccaaaaaatggtctgtcactgcggcgggaagtaaaaataattacgatcactatgaacgaggtgaatacttacacattcgtgattatcaggctgccattgccatcgttgaaaaactatatccagagtatagcacggcaataaaaacgtttaatgatgccagtgatggctattacacaaatatgtttgtcatgcgcaaagatatttttgttgactattctgagtggctcttttccattctggataatctcgaagatgccatctcgatgaacaattataatgctcaggaaaaacgcgttattgggcatatagcagaacggctgtttaatatttacattattaagctgcaacaagatggtgagcttaaggtaaaagaattacagcgtacttttgtcagcaatgaaacattcaatggtgcactgaatccagtttttgattctgcggttccagtggttatcagtttcgatgataattacgcagtcagcggtggtgcattaattaattctattgtccggcatgcggataaaaataaaaattatgatatcgtcgtactcgaaaacaaagtaagctatttgaataaaacgcggttaataaatctaacctcggctcatccgaatatttctcttcgtttttttgacgttaatgccttcactgaaataaacggtgtgcatacccgagcgcattttagcgcatcaacgtatgcccgtctttttattcctcaactgttcagacgatacgataaagtcgtatttattgattcggataccgttgtaaaggctgacctgggtgaactgcttgatgtccctctgggcaacaatttagttgcagcggttaaggatatcgtcatggaaggttttgtaaaattttctgcaatgtcggcatcagatgatggcgttatgccggcaggcgaatatttaaaaaaaaccttaaacatgaataaccctgatgaatattttcaggcagggattattgtttttaatgtcaaacaaatggtcgaagaaaatacttttgctgaattgatgcgggtattaaaggcaaaaaaatactggttcctcgaccaggatatcatgaataaagtcttctactctcgagtcacatttctgccattagagtggaacgtttatcatggtaatggcaacacggatgatttcttccctaatcttaagtttgcaacgtatatgaaatttttagcagctcgcaagaagcctaaaatgattcattatgcgggtgagaacaaaccatggaataccgaaaaagtcgatttttatgacgactttattgaaaacatcgctaacactccatgggagatggaaatctataaacgtcaaatgtcgttagcggcttcgattggtttaacccatagcgagccgcaacaacaaatcttgttccagaccaaaatcaagaacgtactgatgccttatgttaataaatatgcaccaataggcacgccaagaagaaacatgatgactaaatattattacaaagtacgccgtgctattcttggataataaaagagacaacagatgaaaagaaaaaaaatattgatcgtaggcgctggtttctctggtgcagttatcggtcgccaacttgctgagaagggacatcaagtccatattatcgatcagcgtgatcatattggggggaattcctatgatgcacgcgactctgaaacgaatgtgatggtacatgtttatggaccccatattttccatactgacaatgaaacagtgtggaactatgtcaacaagcatgcagagatgatgccctatgtgaaccgggttaaagcgacagttaatggtcaggtattttccctgcctattaatttgcatactatcaatcagtttttctcaaaaacttgttcgcctgatgaggccagagcgctcattgctgagaaaggggacagcactattgctgatccacaaacttttgaagagcaagcgttacgctttattggtaaagagttatatgaggccttttttaaaggatatacgattaaacagtgggggatgcaaccctcggaactgcccgcatctattcttaaacgtcttcctgttcgttttaactatgatgataattattttaaccacaaatttcagggcatgccgaaatgtggttatacgcagatgattaagtcaattctcaatcatgagaatatcaaggttgacttacagcgggaatttatcgttgacgagcgaactcattacgatcacgtattctatagcggtccattagatgcgttttatggctaccaatatggccgtctgggctatcgaacattagattttaaaaagtttatctatcagggtgattaccagggatgcgcagtgatgaactactgttctgtggatgtgccctatactcgcatcactgaacataaatatttttctccctgggaacaacacgacggctctgtttgttataaagagtatagccgtgcttgtgaagaaaatgatattccttactatcctattcgccagatgggagagatggctcttcttgaaaaatatttgtcattggccgagaatgaaaccaacatcacttttgtcggtcgtcttggaacctaccgttaccttgatatggatgtgaccatcgccgaagcattgaaaacggcagaagtctatttaaattcactcactgaaaatcagccaatgcctgtgtttacggtttctgtacgatgaaatatacggcattgatagtgacattcaatcgtctcggcaaactaaaaaaaacggttgaagagaccctcaaacttgaattcactaatattgttattgtcaataacgggtccacggatgggacccaagcctggctttcgtcaattgttgatacacgagtcattgtattaaccctcaccaagaataccggtggggcggggggctttaaaaccggtagtcagtatatctgtgaacagctggcaagtgattgggtatttttctacgatgacgatgcttacccctatccagacacgttgaagtccttttcacagctggataagcagggatgtcgggtatttagtggactggtgaaagatccgcaaggaaaaccgtgtccgatgaatatgccgttctcgcgtgtgccaacttcacttggcgacactgtacgctatttacgctaccctggagagtttatcccggcagctaatcgttctatgttcgtacaaacggtttcatttgttgggatggtcatacatcgtgatctgctcgcgaccagtcttgaccacatccatgaacagctttttatctactttgatgatctttactttggctatcagctatcactagctggtgagaaaattatgtatagcccggagttgcttttttatcatgatgtgagtattcagggcaaacttattgcacctgaatggaaggtttactatctctgccgtaatttgatcctgtcgaagaaaatattccagaaaaatgccgtgtatagcaattcagcgatagcgatacgcatcctaaaatatatattaatcctgccatggcaacgtcaaaaatattcctatatgaaatttattcttcgtggaatttcacatggcataaaaggtattagtggtaagtatcattaagtgggcatagcaatgagaaaattgtgttatttcataaattcggattggtacttcgatttacactggatcgatcgtgccatcgcctcccgtgatgcaggttatgagattcacatcatcagccattttattgatgacaacataataaataaattcaaaacatttggctttatttgccataatgttactcttgatgctcaatcttttaatgcattagttttctttcgtacttaccatgatgtgcaaaaaattattaaaaatataaaaccggatctcttgcattgcatcactatcaagccatgtttgattggtggtgtgctcgcgaagaaatttaatctgccggtcatcgtaagttttgttgggcttggaagagtattttcttctgacagcatgcctttaaaattattgcggcagtttactattgctgcatataaatatattgccagtaataagcgctgtatatttatgtttgaacatgaccgcgacagaaaaaaactggctaagttggttggactcgaagaacaacagactattgttattgatggtgcaggcattaatccagagatatacaaatattctcttgaacaggatcacgatgtccctgttgtattgtttgccagccgtatgttgtggagtaaaggactgggcgacttaattgaagcgaagaaaatattacgcagtaagaatattcactttactttgaatgttgctggaattctggtcgaaaatgataaagatgcaatttcccttcaggtcattgaaaattggcatcagcaaggattaattaactggttaggtcgttcgaataatgtttgcgatcttattgagcaatcaaatatcgttgctttgccgtcagtttattctgaaggtgttccgcgaattcttctggaagcatcttctgtgggtcgcgcttgtattgcttatgatgttggtggttgtgatagccttattattgataacgataatggaattattgttaaaagcaattcacctgaagagctggctgataaacttgcctttttacttagcaatcctaaagcacgcgttgaaatgggtattaaggggaggaaacgtatacaagataaattttctagtgttatgattatcgataaaacattgcaaatatatcatgatgtagttcgatgatgtgtaagtttcacatttattattgcgaaaaaccttcatattgataatagtaatgtttatataatgtaattcaatttactactaatggtatttttatggctcatgaaaaaagtgatataattgtttcggtcgttattcctgtttacaacgccgaagagtatattgcagatactctaaaaaacattgtttcacagtcattgtatgaaattgaaattataataatcaatgatcattcgagtgataatacattagatatccttaaggagattgcatccagcgatgaaagaatacgaattattgataacgctgtaaatattggagctggcatatcacgtaatataggtctttcagaagcaaagggagaatatataatatttcttgatgacgatgattatgtcgatacgaacatgttgaagcacatgtctgattgtgcggagctatcaggggcagatatcgttgtatgcagaagccgctcatttaatctacaatctctccagtatgctccaatgccagattcaattcgaaaagatttattacctgaaaaagcagttttctcgcctggagatattgagcgagactttttcagggcatttatatggtggccatgggacaaactattccgacgtgaatttattattcagcactcgttgagctaccaagatttaagaacatcaaatgatctgttttttgtgtgtgcatctatgcttagtgccgaaaaggtaactattcttgatgaaatattgattactcatacgattaatcgaaaaacatcattgtcttcaactcgctccgtttcctatcattgcgcacttgatgctcttgttgctctaagggattttctttttaaaaatggcatgatgcaaaagcgacaaagggatttttataattacattgtcgtattccttgagtggcacttaaatacgctatcgggtgaagcctttaataaactgtttcaagatgtcaaattattcatcagcagttttgatatcaataatgaagacttttatgatgagtttattctttctgcttatcgacgaatcgctgatatgtctgctgaagagtatcttttttcattaaaagatcgggttattaatgaattagagaatgcccaacgaaatattttgaccttacaaaacgaagttgaggagataaaacagcagcttcaacaaaaggacgaaatgattgcttctatgaatagggaaaatttagctattaaagcagataataaaattctcgaaaattacaatgaagaactaaagactgttcagacaaagtttcttaaactactctcaagtaaagactagtatttaaaagcgtattttatgattactgtaatagcgcccccataaaaaatgagggcggcatagaaattactaataatttatcgttgaccttcgcattgcatctgacgttttaataaccatacaatcatcaatacatcgattggtctcagaaattataacgttgttagatagtttgaaattaaatcctttcataattctgatctcttcagccatacctttgagcgcccagggcgctgctaatgaaatattcccaaactcatcatatctctgtgtttttgtaaaggcattgtaagcaggatctgtgagatcgaacattaattttcctgtgtaattcttaggtattttattagttatttccgcagcaagtgcctgaatttcagagcgttgaggaataataaatccatttataatattatactgagctattatcataattgttaaagcgataagaggccagacaaatgcttgcttagaaattctactgacaaggctatttatgccaataagaaatagagttgatataataagttctaaggccactaacgagcggaatgctgcccaattttcttttgtcgctaaatttggagcgtaggaacctatcccgatcgttatgactatgaacgttttccatctgcctgtttttcccacaaaaatagtgtataagccgattaaaattgcaaatgaggagaaccaagaatatatttttactggttgtatgttatagttatttacagcgtttattagtgattcatttatgaaccatttcatctttccaccgatatctgcggttaactcggctctcgataatgattccccatatagccagacaggaagtacttttgacatgataaaactgcctgcaacaccgataactaaaatgataaaacatgtcgcaacttttttcacagttaaactactttctttttttatgcaactatcaagcataaaaaagaataagaatgtaattgctgtcggttgatatattgcaaatgccacccataagacaacaatggatgctaatttttctggcaatgacgaccgctgcttcgaatgtgggaaacatttattataactaatacctgccagcaatactgaaatagtgaacgggaaacatgttgcccatgaagcataaacttgaaacgcagggagtaagcaaattaacagcggaaatattattttgaatacggggttatcaaatatttttctgctgtctatgaagttgtaaataaaacaacttaagacaacaagacttaatatattaaaaagccgcaaatacgaaaatgaagaaatatcattaattaacatttttccatagtaacggaacacagcataaacgggacgaccagattggacatcccactgaaacgaagagccgtttcttgttatagcatcaaagagtgttgaccagtcgtctgaaaatgcatatgaaaagaaaattaccggtgaaaatgttaacataagcaaaaagaaataaaaaatgtaaacgttttttttatttccctctgctaaaggattgatcagattttgcatgttattttccattgctatcattacctacgctttcgtcaatgaaatatttaggtctatttttcgtctctatataaatttttccgacatattctcctataatacctaaagaaagcatttgcacgccgccaagaaagaatatagcgatcatgactgatgtccatccctcaactgtagtacctgttgttttttgaattaaagcataaatcgcagcgatggtagatatgatgcaagttataaaacctgtcatagctataattcgtaacggtgtaactgataatgaggtaattccctcgagagccagcgcaagcatttttttaattggatattttgattcaccggcaattctttcttcacggctatattgcacctcgatcgaggggtatcccacaagaggcactaatccacgtaaatatatattttgctctttatattgtttaagagcctccaatgctcgattacttaataatcgataatctgcatgatttggagtttgatttactcccaagtgggacattattgcgtaaaatgcattagctgttgtacgtttaaaaaacgtgtcactgtctcgattacctcttacgccgtatactatgtcatatccctggctgtaagcgtcaatcattttttcgatgcaatttacatcgtcttgtagatccgcatcgatgctaatggttacgtctgtatcgaccgagcgtaaccctgccatcaacgcaatttgatgtcctttatttcttgataattttattcctcgcacatagtgataagcggtcgaggcatctttaatttgtgcccaagtattgtcacgactaccatcatcgacaaacaaaagataactattgttattaattttatttctggctatcaatgaatttagtacattcgaaagcttttcgagacagaaaggaaaagcctcttgttcattatagcaaggtaccacaatagctaaagaaggagtgctttttatatcagttgaggttgtcatttcatcgcccagaacttgtttaaaataaaacctgtgatagtgtatgtgaacatcccaaggatttgtgctgaatatattttttctggcataaaaacgaaaaatatttttatgacaatgatatttgccacataacaaatgaagcaaacacataaaaattttattagtctattgatactgattggttgcgtaaatgtaaatattgtgtttgctataaagctgaaaacaatacctacaacataacccatcgcattggacagataaatgccaagacccaaatgcattagcaggaaaattacaactgccgtaattagtgtattgactatcccaactaacccatatttcattagttgccataatgggcctgaacttggcattatatactccgctagcgttccaattggatgttaaaagcggcagcattctaacaaactacatctatcatgtgaatccaattcacatctcaaatattaggttgtaaaggatattgggaggtatttcgagtgctgcgtgaagggttcatttagaaagagtaattaatggcggctttataaccgccatgtcttatattacctatgccgtgctcttaaattatcaatcacttc

SEQ ID NO: 15 - DNA, 3390 bp, Artificial Sequence
  Synthetic nucleotide sequence
tgatttagcactgcactgaatttgggccaggggcaaatctggccgggaactcaaaaatgcatgcaactaaaacagggttatttacagacaaatttaaaattagctgaaagttaatattatttttgcggagccctttcgggccccgaatattactttattttaacattgatttcactttccgggcaacccggcgaaccaggctggtgcctcgttttgcgccttggacatgaattgcttcatacagagcattaaaacggtcatgggcccagccatctctttgctgagaaataagaccatcaaccttattattcaaaagttgtaactcgttactgacagaatgaccaccattgatgctccgcaagcgattcacttcactctcaagttcttgaatcttctcgaggtaggaaggtatcaaccccattgcatagttatatttaacattctcatccataaaaggacggaaactgcggtagttatttttactcttatttccacttaacaacatgccggagtttgcaactctataccaaaatagaggttccgggacgatttgcaatttatatccctgtaatgatattttggcaaaaaactcccagtcttcatgacctaaaccgtaatcttcagtaaatccgcctactttttcgaaaacctcttttctgatcagcgcattagcatcgccaaagcagttactaaagctggcgatatttaaatcaggccctaacggaagccagcagtgcgtcattttacggaacggagaagggaactcctcaccaaaaataagatcgcttggtgtggttaacacatcggccccagagtttaatgctgcagtaacaaacgtttctacctcaaaaggcttagcaacattatcatcgtccataaacatcagatattcgccagaggcgtgtcgcgcagccaaattccttgcagcacccagatagttattagaactacggacaattttccagcctcgagagttaaaatcattctcgatgagattcaaataacgatgagaatcttctgtcgtacttccatcatcaaccaagatgacctcaatattttggtacgtctgagattttattgatgcgagtgcttgctgaagcaaatggtgacgttcgaagtgagttatacacacgctaactaacgggctgttagcttcatcgattttcttgaatgcgcggttgttttttcgttcaactgcgacaaaccaagcttctttaatattgtcttgtgattcagcaagccctggttttatatttatattttttaagcgatagtggattttcccgtataaatcgacaggtgtaggaataaatagaacttccgcatgatgctcctgcggaataagctctggaattccaccaacgtttgaagcgaggaaattaacgttattaatcaagcattcataaacagtatagggtgagttttctacaagtgatggaatgatgactaatacattttttctttttatatattcattagcgttggtacgatcatagtcgctgatgacattaactgcgagtcccaaatttttagtctgattcataatataagtaaatgaatcagttttccccatagtgacattttttccgaggaaggttactccagaaatgctctctttatcttcatcagatagttttcttaatgcacgcaggaatatgtcaagtcctttacgggtttcaaggcggccgaaaaatacaagctcaacgccagaagctggcttttcatttattttaactgtaacatcatctctcgtcacaaacccttgaaatggctcgcaatttaaaattacatgacgttcttcaggaacattccagtgcttactcaacatccaatcaattaaatactgagacggactaacaacttcatccgccatttcaaccaccattttctccatataatagagttcaagatggttctgatcatatggaagctggtaattaccttcatcagcccataacgttgaactgtgagtatttacaatgaactttgtattttcaaaatccgttccattctttttgcttaataaagtgtaataaagatctgcctgccactcacaagaaataacagtgtcatagatgttattttctttcaaccagagataaattgaataacttttccttctaaaatacggtgcatcaatattaatctcttttatcagtccggttcttagcagattgataccaaaggtactataaatacgtgaccagtcgctaaatttcgatacagatgattcagaatagtcgccacatgtatacaatacatcaacatcataccccttttttgccaaagtagtggcaagggcagtgaaagcagttccaataccgccgttacggacaggcccctcaatgtccggcgtcattataagaattttcttcattgtaacccttcctttgtaacctagacttttctatgatattagtgaattgaagtagtgtaagatagcagtcggtagcttctgttaaacaggataaaaaatgaccaatatgaagttaaaatttgatttgcttctaaaatcttatcatctatctcatcgatttgtctataaggcaaaccctggtaatgctggtgatggtgtaattgcatctgcgacatatgacttttttgaacgaaatgctcttacctatatcccttacagagatggcgagcgctacagttctgaaactgatattttaatttttggaggcggaggaaacctgatagaaggattgtattctgaaggtcatgactttatccagaataatattgggaagtttcataaagtaataataatgccgtcgacaatcagagggtatagcgatttattcatcaacaatattgataagtttgttgttttttgtcgcgaaaatatcaccttcgattatattaaatctctcaactacgaaccaaacaagaacgtattcattactgatgatatggcattttatctcgatcttaataaatacctgtcacttaaacccatctataaaaaacaggccaactgcttcagaacggactccgaatctctaactggagactataaagaaaacaatcatgatatttcgctcacctggaatggcgattattgggataatgaatttctggcgcgtaattctacccgttgcatgataaactttcttgaagagtataaagttgtcaataccgacaggctgcatgtggcaattttagcatctctgcttggcaaagaagtcaacttctatcctaactcatattacaaaaatgaagctgtttacaattattcactttttaatcgttatccaaaaacatgctttattacggcaagttgaaaaaggcagcgtataataatacgctgcctgaaagccatataactgttacagcattgttaattattgcctgccagcctttaggtgactattcattcgcacgcctata

SEQ ID NO: 16 - DNA, 33 bp, Artificial Sequence
  Synthetic nucleotide sequence
atgagtataaagatgaagtacaatttagggtat

SEQ ID NO: 17 - DNA, 30 bp, Artificial Sequence
  Synthetic nucleotide sequence
gaagtgattgataatttaagagcacggcat

SEQ ID NO: 18 - DNA, 53 bp, Artificial Sequence
  Synthetic nucleotide sequence
atgagtataaagatgaagtacaatttagggtatttatttgatttacttgttgt

SEQ ID NO: 19 - DNA, 34 bp, Artificial Sequence
  Synthetic nucleotide sequence
ggaagtgattgataatttaagagcacggcatagg

SEQ ID NO: 20 - DNA, 88 bp, Artificial Sequence
  Synthetic nucleotide sequence
caacataggaggaaattatatgagtataaagatgaagtacaatttaggggtttaaaccctatgccgtgctcttaaattatcaatcaca

SEQ ID NO: 21 - DNA, 96 bp, Artificial Sequence
  Synthetic nucleotide sequence
agcttgtgattgataatttaagagcacggcatagggtttaaacccctaaattgtacttcatctttatactcatataatttcctcctatgttgagct

SEQ ID NO: 22 - DNA, 92 bp, Artificial Sequence
  Synthetic nucleotide sequence
ctagcaacataggaggaaattatatgagtataaagatgaagtacaatttaggggtttaaaccctatgccgtgctcttaaattatcaatcaca

SEQ ID NO: 23 - DNA, 92 bp, Artificial Sequence
  Synthetic nucleotide sequence
agcttgtgattgataatttaagagcacggcatagggtttaaacccctaaattgtacttcatctttatactcatataatttcctcctatgttg

SEQ ID NO: 24 - DNA, 26 bp, Artificial Sequence
  Synthetic nucleotide sequence
tgatttagcactgcactgaatttggg

SEQ ID NO: 25 - DNA, 27 bp, Artificial Sequence
  Synthetic nucleotide sequence
tataggcgtgcgaatgaatagtcacct